(19) United States
(12) Patent Application Publication
(10) Pub. No.: US 2005/0013885 A1
(43) Pub. Date: Jan. 20, 2005

(54) Extracts derived from chenopodium plants and uses thereof

(76) Inventor: Helene Chiasson, Saint-Jean-sur-Richelieu (CA)

Correspondence Address: NEEDLE & ROSENBERG, P.C., SUITE 1000, 999 PEACHTREE STREET, ATLANTA, GA, 30309-3915, US

(21) Appl. No.: 10/467,696

(22) PCT Filed: Jul. 14, 2003
(86) PCT No.: PCT/CA03/01002

Related U.S. Application Data
(63) Continuation-in-part of application No. 10/195,131, filed on Jul. 12, 2002.

(30) Foreign Application Priority Data
Jul. 12, 2002 (US) 10/195,131

(51) Int. Cl. A61K 35/78; A01N 65/00

(52) U.S. Cl. 424/764

(57) ABSTRACT

The present invention relates to pesticides. More particularly, the present invention relates to botanical pesticides. In particular, the present invention relates to compositions and methods for controlling plant-infesting pests with plant extracts and notably with compositions comprising oil extracts derived from Chenopodium sp. plant material. The invention further relates to compositions comprising such extracts as pesticidal compositions and providing the advantages of minimal development of resistance thereto, minimal toxicity to mammals, minimal residual activity and environmental compatibility. The pesticidal compositions of the present invention comprises α-terpinene, ρ-cymene, limonene, carvacrol, carveol, nerol, thymol, and carvone.

### FIELD OF INVENTION

The present invention relates to the field of pesticides for controlling plant-infesting pests.

### BACKGROUND OF THE INVENTION

Plant feeding mites are among the most voracious phytophagous pests of crops. To combat these pests, synthetic pesticides have been developed. These synthetic chemical pesticides, however, often have detrimental environmental effects that are harmful to humans and other animals and therefore do not meet the guidelines developed by most Integrated Pest Management programs. Moreover, resistance to these products has been found to develop with many of the new products put on the market (Georghiou, 1990; Nauen et al., 2001).

Although resistance follows a highly complex genetic and biochemical process, it can generally develop rapidly with synthetic products because their active ingredients rely on one or more molecules of the same class. The organism can therefore respond to the toxin by developing physiological, behavioral or morphological defense mechanisms to neutralize the effect of the molecule (Roush and MacKenzie, 1987).

Acari, such as spider mites, in particular, are extremely difficult to control with pesticides. Tetranychus urticae (the two-spotted spider mite), for example, has accumulated a considerable number of genes conferring resistance to all major classes of acaricides. Resistance to many registered acaricides have been reported, for example, resistance has been reported to hexythiazox, abamectin, and clofentezine. Furthermore, many of these pesticides have been found to exacerbate pest infestation by destroying the natural predators of mites (U.S. Pat. No. 5,839,224). Additionally, many synthetic insecticides have been found to stimulate mite reproduction. For example, it was found that mites reproduce many times faster when exposed to carbaryl, methyl parathion, or dimethoate in the laboratory than untreated populations (Flint, 1990).

As a result, there are very few pesticides remaining that are effective against spider mites (Georghiou, 1990). In the Farm Chemical Handbook (Meister, 1999), for example, only 48 products out of a total of 2,050 listed acaricides and insecticides (or 2.4%), were identified as acaricides and only 69 of these products (or 3.4%) were identified as both acaricides and insecticides.

The control of insect infestation has also proven to be difficult. For example, European chafer Rhizotrogus majalis Razoumowsky (Coleoptera: Scarabaeidae) insects are an exotic species to Canada and the United States and are commonly known as Scarab beetles. Throughout the northeast, where the beetle has become established, R. majalis causes major damage to turfgrass, consuming the roots of home lawns as well as urban recreational areas and golf courses. The larvae of this species are extremely difficult and expensive to control using conventional insecticides. In several areas of the U.S. insecticide resistance has already developed within populations.

The control of plant fungi is another source for growing concern. Phytopathogenic fungi are of great economic importance since fungal growth on plants or on parts of plants inhibits production of root, stem, foliage, fruit or seed, and the overall quality of a cultivated crop. About 25 percent of all fungal diseases in agriculture and horticulture are caused by powdery mildew phytopathogens (U.S. Pat. Nos. 5,882,689 and 5,496,568). In cucumber crops, for example, powdery mildew caused by the plant pathogens, Erysiphe cichoracearum and Sphaerotheca fuliginea, is one of the most problematic disease.

The fungus responsible for Gray Mold, Botrytis cinerea, can attack more than 200 species of cultivated plants and especially those growing in a greenhouse environment. It is a saprophyte that attacks dead or senescent plant tissue. B. cinerea is especially damaging to stems (causing stem rot), when it enters scars left by pruning of lower leaves. Following attack, the plant dies, causing heavy economic losses to the grower. Control of stem rot is attempted by treating leaf scars after leaf pruning. Although benomyl is presently used, it will likely be removed from the market shortly. Moreover, iprodione (another pesticide), is less effective because of developing fungal resistance.

In spite of their strong fungicidal effect, agricultural fungicides currently in use have been a source of problems, because the required amount is ever increasing as tolerance of target plant pathogens increases. Moreover, many fungicides are synthetic agents, which when used, pose possible drawbacks for humans, animals and the environment. Thus, effective commercial products (to combat fungal attacks on crops), which reduce the dose of synthetic chemicals spread into the environment, are in need; keeping in mind a regard for environmental and human safety issues.

As an alternative to synthetic agents, botanical pesticides offer the advantage of being naturally derived compounds that are safe to both humans and the environment. Specifically, botanical pesticides offer such advantages as being inherently less toxic than conventional pesticides, generally affecting only the target pest and closely related organisms, and are often effective in very small quantities. In addition, botanical pesticides often decompose quickly and, therefore, are ideal for use as a component of Integrated Pest Management (IPM) programs.

There are few published reports of the acaricidal properties of botanical pesticides. For example, U.S. Pat. No. 4,933,371 describes the use of saponins extracted from various plants (i.e., yucca, quillaja, agave, tobacco and licorice) as acaricides. This patent also describes the use of linalool extracted from the oil of various plants such as Ceylon's cinnamon, sassafras, orange flower, bergamot, Artemisia balchanorum, ylang ylang, rosewood and other oil extracts as acaricides. These methods, however, require the extraction of one active substance from the plant which often does not meet desired levels of toxicity towards acari.

Plant essential oils are a complex mixture of compounds of which many can be biologically active against insect and mite pests, the compounds acting individually or in synergy with each other, to either repel or kill the pests by contact. These components are plant secondary metabolites or allelochemicals produced by plants as a defense mechanism against plant feeding pests (Ceske and Kaufman, 1999). Because of the complexity of the mixture, it has been observed that pests do not easily develop resistance to these products as they can to synthetic pesticides or botanical pesticides comprising a single active compound. In this respect, Feng and Isman (1995) demonstrated that repeated treatments of pure azadirachtin, a major active constituent of neem oil, against the green peach aphid led to a 9-fold resistance after 40 generations. However, repeated exposure during 40 generations to crude neem extracts did not lead to resistance.

There remains a need to provide new and effective pesticidal products which overcome the shortcomings of products known in the art. For example, there remains a need for broad spectrum compositions which are less likely to enable pests to develop resistance thereto. There also remains a need to provide a method to combat pests at any given locus, using a composition which is not toxic to animals, especially to mammals, nor to any beneficial predator/parasitoid insects.

### SUMMARY OF THE INVENTION

The invention provides essential oil extracts derived from Chenopodium sp. comprising, α-terpinene, ρ-cymene, limonene, carvacrol, carveol, nerol, thymol, and carvone, wherein these extracts have one or more activities selected from the group of acaricidal, pesticidal, insecticidal, and fungicidal activities. One or more of these extracts can be formulated into compositions that enable their application into different environments such as plants, soil, animals and buildings.

If accordance with another aspect of the invention there is provided composition comprising one or more Chenopodium sp. -derived essential oil extracts, wherein said extract comprises α-terpinene, ρ-cymene, limonene, carvacrol, carveol, nerol, thymol, and carvone, in combination with an emulsifier, carrier, spreader and/or sticking agent, to enable application of the composition to a specific environment, wherein the composition has one or more activities selected from the group of acaricidal, pesticidal, insecticidal, and fungicidal activities.

### BRIEF DESCRIPTION OF THE DRAWINGS

FIG. 1 shows the chemical content of three lots or pools of oil samples extracted from whole plant parts above root (00MC-21P, 00MC-24P and 00M-29P).

FIG. 2 shows the average mortality (%) of the two-spotted spider mite (TSSM: Tetranychus urticae) when tested with solutions of individual compounds present in the essential oil of Chenopodium ambrosioides. Results adjusted for control mortality with Abbott's formula.

FIG. 3 shows the average mortality (%) of the greenhouse whitefly (GWF; Trialeurodes vaporaiorum) when tested with solutions of individual compounds present in the essential oil of Chenopodium ambrosioides. Results adjusted for control mortality with Abbott's formula.

FIG. 4 shows adult spider mite (Tetranychus urticae) mortality obtained with bioassays using the RTU formulation of Chenopodium ambrosioides and commercial preparations of natural and synthetic insecticides.

FIG. 5 shows spider mite egg (Tetranychus urticae) mortality, using the RTU formulation of Chenopodium ambrosioides oil.

FIG. 6 shows spider mite nymph (Tetranychus urticae) mortality, using the RTU Chenopodium extract formulation and commercial preparations of synthetic and natural products.

FIG. 7 shows the mortality of adult spider mites 48 h following introduction on faba bean leaves treated one hour previously with the RTU formulation and selected natural acaricides.

FIG. 8 shows red mite, Panonychus ulmi mortality, using the RTU formulation.

FIG. 9 shows insect mortality (%) obtained with bioassays using the RTU formulation of Chenopodium ambrosioides.

FIG. 10 shows mortality of adult female twospotted spider mites 48 hours following applications.

FIG. 11 shows mortality of adult female European red mite 24 hours following applications.

FIG. 12 shows egg hatch (%) of the twospotted spider mite, 10 days following applications.

FIG. 13 shows egg hatch (%) of European red mite 10 days following applications.

FIG. 14 shows mortality of adult female two-spotted spider mites 48 hours following introduction on leaf discs treated with EC25% and Dicofol one hour previously.

FIG. 15 shows mortality of green peach aphids (Myzus persicae (Sulz.)) 48 hours following application of 0.125, 0.25, 0.5, 1.0 and 2.0% concentrations of formulation EC25% and the commercially available bioinsecticides Neem Rose Defense® and Safer's Trounce®

FIG. 16 shows lethal concentrations (LC₅₀ and LC₉₀) in % of EC25% for the green peach aphid (Myzus persicae (Sulz.)) calculated with 48 hour mortality data.

FIG. 17 shows average number of green peach aphids (Myzus persicae (Sulz.)) per cm² of treated Verbena speciosa shoot following application of 0.25, 0.50 and 1.0% concentrations of EC25% and the commercially available bioinsecticides Neem Rose Defense® and Safer's Trounce®

FIG. 18 shows mortality of Western flower thrips (Frankliniella occidentalis (Perg.)) 24 hours following application of six concentrations (0.05, 0.125, 0.18, 0.25, 0.5 and 1.0%) of formulation EC25% and the commercially available bioinsecticides Neem Rose Defense® and Safer's Trounce®

FIG. 19 shows lethal concentrations (LC₅₀ and LC₉₀) in mg/cm² of EC25% for the Western flower thrips (Frankliniella occidentalis (Perg.)) calculated with 24 hour mortality data.

FIG. 20 shows average number of Western flower thrips/cm² (WFT: Frankliniella occidentalis (Perg.)) per treatment as a percentage of thrips present on leaves treated with the control during a greenhouse bioassay using two concentrations (0.25 and 1.0%) of EC25% and two commercially available bioinsecticides Neem Rose Defense® and Safer's Trounce®

FIG. 21 shows mortality of greenhouse whiteflies (Trialeurodes vaporariorum (Westw.)) 20 hours following application of five concentrations (0.0625, 0.125, 0.25, 0.5 and 1%) of formulation EC25% and the commercially available insecticides Neem Rose Defense®, Safer's Trounce® and Thiodan®

FIG. 22 shows lethal concentrations (LC₅₀ and LC₉₀) in mg/cm² of EC25% for the greenhouse whitefly (Trialeurodes vaporariorum (Westw.)) calculated with 20 hour mortality data.

FIG. 23 shows mortality of Encarsia formosa 24 hours following application of four concentrations (0.0625, 0.125, 0.25, 0.5 and 1.0%) of formulation EC25% and the commercially available bioinsecticides, Neem Rose Defense® and Safer's Trounce®

FIG. 24 shows the effect of three concentrations of EC25% with the tomato canker caused by the fungus Botrytis cinerea.

FIG. 25 shows the percentage control of the tomato canker following treatment with a selection of products 1 and 8 days following inoculation with Botrytis cinerea.

FIG. 26 shows the percentage (%) infection among blocks with powdery mildew.

FIG. 27 shows the effect of EC25% and reference products on Powdery Mildew.

FIG. 28 shows daily mean and total emergence of adult thrips from soil treated with different volumes of EC25% at 0.5% concentration.

FIG. 29 shows the percent survival of R. majalis 7 days after treatment with EC25%.

FIG. 30 shows EC25% median lethal concentrations (LC₅₀), standard error of log LC₅₀, 95 percent confidence interval and slope/intercept equation for two 7 day trials with R. majalis.

FIG. 31 shows average soil percent organic matter and pH±standard error determined for each tray in both R. majalis 7 day trial.

FIG. 32 shows treatments tested with the European chafer, Rhizotrogus majalis.

FIG. 33 shows counts for live European chafers on turfgrass plots.

FIG. 34 shows treatments evaluated with the HCB on turfgrass plots.

FIG. 35 shows counts for live HCB on turfgrass plots.

FIG. 36 shows mean mortality (%) of Amblyseius fallacis adult females following the direct application of several concentrations of EC25% and commercially available insecticides.

FIG. 37 shows contact toxicity of EC25% oil formulation on adult females of Amblyseius fallacis. Probit analysis.

FIG. 38 shows mean percent mortality of Phytoseiulus persimilis adult females to different insecticide treatments.

FIG. 39 shows overall percent mean mortality of adult wasps Aphidius colemani following direct application with EC25% and commercially available insecticides.

FIG. 40 shows male and female mean mortality (%) of Aphidius colemani adult wasps following direct application with EC25% and commercially available insecticides.

FIG. 41 shows contact toxicity of EC25% oil formulation on adult wasps Aphidius colemani. Probit analysis.

FIG. 42 shows mortality of adult wasps Aphidius colemani following exposure to EC25% and commercially available insecticide residues.

FIG. 43 shows probit analysis of adult wasps Aphidius colemani 24 H and 48 H following exposure to EC25% residues.

FIG. 44 shows the effect of treatment on Aphidius colemani emergence from treated mummies.

FIG. 45 shows fecundity assessment of female Aphidius colemani following contact with EC25% residues.

FIG. 46 shows mean mortality of Orius insidiosus second instar nymphs following application with EC25% and commercially available insecticides.

FIG. 47 shows mean mortality of Orius insidiosus adults following EC25% and other insecticide treatments.

FIG. 48 shows fecundity of Orius insidiosus females surviving insecticide treatments.

FIG. 49 shows probit analysis of Orius insidosus second instar nymphs following application with EC25%.

FIG. 50 shows probit analysis of Orius insidiosus adults following application with EC25%.

### DETAILED DESCRIPTION OF THE INVENTION

Definitions

Unless defined otherwise, all technical and scientific terms used herein have the same meaning as commonly understood by one of ordinary skill in the art to which this invention belongs.

“Locus” means a site which is infested or could be infested with acari, insects, fungi, or other pests and may include, but is not restricted to, domestic, urban, agricultural, horticultural, and forest environments.

“Essential Oil Extract” means the volatile, aromatic oils obtained by steam or hydro-distillation of plant material and may include, but are not restricted to, being primarily composed of terpenes and their oxygenated derivatives. Essential oils can be obtained from, for example, plant parts including, for example, flowers, leaves, seeds, roots, stems, bark, wood, etc.

“Active Constituents” means the constituents of the essential oil extract to which the pesticidal, acaricidal, insecticidal, and/or fungicidal activity is attributed. The essential oil extract of the present invention generally comprises the active constituents including: α-terpinene, ρ-cymene, limonene, carvacrol, carveol, nerol, thymol, and carvone.

The term “partially purified”, when used in reference to an essential oil extract means that the extract is in a form that is relatively free of proteins, nucleic acids, lipids, carbohydrates or other materials with which it is naturally associated in a plant. As disclosed herein, an essential oil extract of the invention is considered to be partially purified. In addition, the individual components of the essential oil extract can be further purified using routine and well known methods as provided herein.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference.

The present invention provides essential oil extracts derived from Chenopodium sp. comprising, α-terpinene, ρ-cymene, limonene, carvacrol, carveol, nerol, thymol, and carvone. These extracts have one or more activities selected from the group of acaricidal, pesticidal, insecticidal, and fungicidal activities.

The present invention also provides for the use of one or more of these essential oil extracts to prepare compositions that enable their application into different environments such as plants, soil, animals and buildings.

1. Essential Oil Extract

Plant Material

Plant material that may be used in the present invention includes plant material derived from the genus Chenopodium sp. taken individually or in a group and may include, but is not restricted to, the leaf, flowers, roots, seeds, and stems. As is known by persons skilled in the art, the chemical composition and efficacy of an essential oil extract varies with the phenological age of the plant (Jackson et al., 1994), percent humidity of the harvested material (Chialva et al., 1983), the plant parts chosen for extraction (Jackson et al., 1994; and Chialva et al., 1983), and the method of extraction (Perez-Souto, 1992). Methods well-known in the art can be adapted by a person of ordinary skill in the art to achieve the desired yield and quality of the essential oil extract of the present invention. In one embodiment, the plant material is derived from Chenopodium ambrosioides.

Pre-Treatment of Plant Material

In addition to such parameters as the phenological age of the plant, the percent humidity of the harvested material, the plant parts chosen for extraction, and the method of extraction, the chemical composition and efficacy of an essential oil extract may be affected by pre-treatment of the plant material. For example, when a plant is stressed, several biochemical processes are activated and many compounds, in addition to those constitutively expressed, are synthesized as a response. In addition to pests, fungi, and other pathogenic attacks, stressors include drought, heat, water and mechanical wounding. Moreover, persons of skill in the art will also recognize that combinations of stressors may be used. For example, the effects of mechanical wounding can be increased by the addition of compounds that are naturally synthesized by plants when stressed. Such compounds include jasmonic acid (JA). In addition, analogs of oral secretions of insects can also be used in this way, to enhance the reaction of plants to stressors.

In one embodiment, the essential oil extracts of the present invention are derived from plant material which has been pre-treated, for example by stressing the plant by chemical or mechanical wounding, drought, heat, or cold, or a combination thereof, before plant material collection and extraction.

Harvesting the Plant Material for Extraction and Optional Storage Treatment

The plant material may be used immediately after harvesting. In one embodiment the fresh plant material having a humidity level of >75% is used. Otherwise, it may be desirable to store the plant material for a period of time, prior to performing the extraction procedure(s). In another embodiment wilted plant material having a humidity level of 40 to 60% is used. In another embodiment dry plant material having a humidity level of <20% is used. In a further embodiment, the plant material is treated prior to storage. In such cases, the treatment may include drying, freezing, lyophilisizing, or some combination thereof.

Extraction of the Essential Oil Extract and Validation of Constituents

Essential oil extracts can be extracted from plant material by standard techniques known in the art. A variety of strategies are available for extracting essential oils from plant material, the choice of which depends on the ability of the method to extract the constituents in the extract of the present invention. Examples of suitable methods for extracting essential oil extracts include, but are not limited to, hydro-distillation, direct steam distillation (Durbeck 1997), solvent extraction, and Microwave Assisted Process (MAP™) (Belanger et al., 1991).

In one embodiment, plant material is treated by boiling the plant material in water to release the volatile constituents into the water which can be recovered after distillation and cooling. In another embodiment, plant material is treated with steam to cause the essential oils within the cell membranes to diffuse out and form mixtures with the water vapor. The steam and volatiles can then be condensed and the oil collected. In another embodiment, organic solvents are used to extract organically soluble compounds found in essential oils. Non-limiting examples of such organic solvents include methanol, ethanol, hexane, and methylene chloride. In a further embodiment, microwaves are used to excite water molecules in the plant tissue which causes cells to rupture and release the essential oils trapped in the extracellular tissues of the plant material.

To confirm the presence of the constituents of the present invention in the essential oil extract, a variety of analytical techniques well known to those of skill in the art may be employed. Such techniques include, for example, chromatographic separation of organic molecules (e.g., gas chromatography) or by other analytical techniques (e.g., mass spectroscopy) useful to identify molecules falling within the scope of the invention.

The invention provides an essential oil extract derived from Chenopodium sp. comprising α-terpinene, ρ-cymene, limonene, carvacrol, carveol, nerol, thymol, and carvone.

In one embodiment, the essential oil extract comprises at least 30% α-terpinene, 8% ρ-cymene, 5% limonene, trace carvacrol, 0.1% carveol, 0.1% nerol, trace thymol, and trace carvone.

In one embodiment, the essential oil extract comprises at least one of each compound selected from the range for each compounds of at least 30%, 32%, 34%, 38%, 40%, 44%, 48%, 50%, 55%, 60 %, 65% α-terpinene; 8%, 10%, 12%, 14%, 16%, 18%, 20%, 22%, 24%, 26% ρ-cymene, 5%, 6%, 7%, 8%, 9%, 10%, 12%, 14%, 16%, 18%, 20%,22%,24% limonene, trace, 0.04%, 0.08%, 0.10%, 0.12%, 0.14%, 0.2%, 0.5%, 0.8%, 1.0% carvacrol, 0.1%, 0.2%, 0.4%, 0.6%, 0.8%, 1.0%, 1.4%, 1.6%, 1.8%, 2.0% carveol, 0.1%, 0.2%, 0.4%, 0.6%, 0.8%, 1.0%, 1.4%, 1.6%, 1.8%, 2.0% nerol, trace, 0.04%, 0.08%, 0.10%,0.12%, 0.14%, 0.2%, 0.5%, 0.8%, 1.0%, 1.4%, 1.6%, 1.8%, 2.0% thymol, and trace, 0.04%, 0.08%, 0.10%, 0.12%, 0.14%, 0.2%, 0.5%, 0.8%, 1.0% carvone.

2. Activity of the Essential Oil Extract

Following extraction of an essential oil extract of the invention, it may be desirable to test the efficacy of the extracts for acaricidal, pesticidal, insecticidal, and fungicidal activities. Any number of tests familiar to a worker skilled in the art may be used to test the activity of the extracts, compositions, and formulations of the invention.

Deternination of Acaricidal Activity of the Essential Oil Extract

Acaricidal activity of an essential oil extract may be evaluated by using a variety of bioassays known in the art (Ebeling and Pence, 1953; Ascher and Cwilich, 1960; Dittrich, 1962; Lippold, 1963; Foot and Boyce, 1966; and Busvine, 1980).

a) Contact Efficacy with the Adult Stage

One exemplary method that may be used tests the contact efficacy of the essential oil extract, or formulations thereof, with the adult stage of a mite species. For example, adult mites may be placed on their dorsum with a camel hair brush on a double-sided sticking tape glued to a 9 cm Petri dish. Essential oil extracts and/or formulations may then be applied to the test subjects by spraying with the spray nozzle of a Potter Spray Tower mounted on a stand and connected to a pressure gauge set at 3 psi. Mites that fail to respond to probing with a fine camel hair brush with movements of the legs, proboscis or abdomen are considered dead.

In one embodiment, the contact efficacy of an essential oil extract, or formulations thereof, is determined using the two-spotted spider mite (Tetranychus urticae), at the adult stage, as a model test subject. A person skilled in the art, however, will readily understand that other species of acari can be used.

b) Ovicidal Activity

The ovicidal effect can be determined by treating mite eggs with concentrations of essential oil extracts, or formulations thereof. For example, adult female T. urticae may be transferred to 2 cm diameter leaf disks cut out of lima bean leaves and left for four hours for oviposition. When at least 20 eggs/disk are laid, adult mites may then be removed. Essential oil extracts and/or formulations may then be applied by spraying the test subjects. Egg hatch is assessed daily and for 10 days following treatment by counting the number of eggs remaining on the leaf disks and the number of live and dead nymphs present. Percent egg hatch is determined with live nymphs only. The nymphs are considered dead if no movement is observed after repeated gentle probing with a single-hair brush.

In one embodiment the ovicidal activity of an essential oil extract, or formulations thereof, is determined with mite eggs of the two-spotted spider mite (Tetranychus urticae), as a model test subject. A person skilled in the art, however, will readily understand that other species of acari can be used.

Determination of Insecticidal Activity of the Essential Oil Extract

Similar bioassays can be conducted to evaluate the insecticidal activity of an essential oil extract, or formulations thereof, by utilizing an insect model. In one embodiment, the greenhouse whitefly (Trialeurodes vaporariorum (Westw.)) is used as a model test subject in an insecticide bioassay. For example, Whitefly adults may be glued to a black 5 cm×7,5 cm plastic card sprayed with Tangle-Trap® (Gempler's Co.) to obtain at least 20 active adults per card. Each card is sprayed with the essential oil extract, composition, or formulation and allowed to dry. The cards are then placed sideways on a Styrofoam rack in a closed clear plastic container of 5 L with moistened foam on the bottom to keep humidity high (>90% R.H.). The plastic container is stored in a growth chamber at 24° C. and 16 L:8 D photoperiod. Mortality is evaluated 20 hours following treatment by gently probing the whitefly with a single-hair brush under the binocular microscope. Absence of movement (antennae, leg, wing) following probing is recorded as dead. A person skilled in the art, however, will readily understand that other insect species can be used.

Determination of Fungicidal Activity of the Essential Oil Extract

The fungicidal activity of an essential oil extract, or formulations thereof, can also be evaluated by utilizing a fungal model evaluated by using a variety of bioassays known in the art. Examples of such known bioassays include the following.

Laboratory Tests

The fungicidal efficacy of an essential oil can be done in the laboratory using several methods. One method incorporates the test samples in an agar overlay in a Petri dish. A second method would use a filter disk saturated with the test samples and placed on top of untreated agar. Both systems are challenged with fungal plugs cut from lawns of indicator organisms at the same stage of growth. The plates will be incubated at 30° C. for 5-10 days with visual observations and the zone of inhibition measured and recorded. A positive control, i.e. a commercially available fungicide and a negative control, i.e. water are tested in the same way.

Greenhouse Tests

Greenhouse tests may also be employed to evaluate fungicidal efficacy. For example, the effect of the essential oil extracts, or formulations thereof, may be tested on host plants infected by a disease organism such as, for example, Botrytis cinerea, Erysiphe cichoracearum or Sphaerotheca fuliginea, Rhizoctonia solani, and Phytophthora infestans, by observing the percent damage or presence of lesions on the host plant after treatment and against controls.

Botrytis cinerea. Tomato plants are seeded and grown following current commercial practices for greenhouse tomato production. About 2 months following seeding, lesions are made on the leaves and the stem (5 lesions/plant) and inoculated with a suspension of 3×10⁶ spores of B. cinerea, 2 ml per lesion. Treatments are then applied to the plants. A positive control, i.e. a commercially available fungicide and a negative control, i.e. water are also tested and all treatments are done in a randomized block design.

The length of lesions are measured every two weeks over a period of 3 months, then the number of fruit, the total weight of fruit and the average weight of fruit are calculated during the entire production period of the plant. The experiment is repeated and the effect of treatments is subjected to an analysis of variance (ANOVA) and means are compared with a LSD test.

Erysiphe cichoracearum or Sphaerotheca fuliginea. These disease organisms are obligatory parasites that do not have the capacity to survive in absence of its host. Therefore to provide the inoculum for the test, cucumber leaves are taken from an infested greenhouse. The conidia present on these leaves will transfer onto cucumber plants grown for the experiment one or two months previously. New plants are periodically infested in this manner in order to increase the inoculum.

Treatments are then applied to the plants before or after inoculation depending on the type of fungicide used. A positive control, i.e. a commercially available fungicide and a negative control, i.e. water are also tested and all treatments are done in a randomized block design.

The effect of the disease is evaluated on individual leaves of all plants using a index of infestation from 0 to 5 (0=absence of blemish and 5=80-100% of the leaf surface with blemishes). The degree of the infestation is evaluated 3, 7, and 14 days following inoculation and reported in averages per plant. The experiment is repeated and the effect of treatments is subjected to an analysis of variance (ANOVA) and means are compared with a LSD test.

Rhizoctonia solani. An isolate of Rhizoctonia solani is produced on a culture media (PDA) 3 days before inoculation and a plug of the disease is then transferred to Erlenmeyer flasks filled with a YMG broth for 5 days. The mycelium is filtered, suspended in distilled water and blended. Seeds of tomato are used and sterilized on the surface using successive ethanol 70%, bleach and distilled water solutions. A suitable sterile potting soil mix is used in which 60 mg blended mycelium is inoculated per 100 g of potting soil.

Tests are done in bedding boxes of 72 cells/box and 3 boxes are used per treatment. The boxes are spread out in a randomized arrangement in a controlled atmosphere growth chamber the following conditions: 20° C. during the day and 16° C at night, 16 hours of light, 162 umol of light intensity and 60% humidity. The boxes are incubated in the growing chambers during 3 weeks.

Treatments are then applied to the young plants before or after inoculation depending on the type of fungicide used. A positive control, i.e. a commercially available fungicide and a negative control, i.e. water are also tested and all treatments are done in a randomized block design.

Plants are examined each week and the incidence of the disease is measured as well as the degree of infestation on a scale of 0 to 5 (0=absence of infestation and 5=80-100% of the leaf surface attacked). The experiment is repeated and the effect of treatments is subjected to an analysis of variance (ANOVA) and means are compared with a LSD test.

Phytophthora infestans. On tomato plants. Tomato plants are seeded and grown following current commercial practices for greenhouse tomato production. About 2 months following seeding, leaves and stems are inoculated with a suspension of 1×10⁴ spores of P. Infestans until the plant surfaces are completely covered. Treatments are then applied. A positive control, i.e. a commercially available fungicide and a negative control, i.e. water are also tested and all treatments are done in a randomized block design.

Percent damage or presence of lesions is evaluated every 3-4 days for a period of 2 weeks on leaves that had been identified previously (15-30 leaves per plant). The experiment is repeated and the effect of treatments is subjected to an analysis of variance (ANOVA) and means are compared with a LSD test.

On potato plants. Potato tubers are sown and grown in pots of 6-8 inches. About 1,5 months after seeding, the leaves and stems of the plants are inoculated with a suspension of 1×10⁴ spores of P. Infestans until the plant surfaces are completely covered. Treatments are then applied. A positive control, i.e. a commercially available fungicide and a negative control, i.e. water are also tested and all treatments are done in a randomized block design.

Percent damage or presence of lesions is evaluated every 3-4 days for a period of 2 weeks on leaves that had been identified previously (15-30 leaves per plant). The experiment is repeated and the effect of treatments is subjected to an analysis of variance (ANOVA) and means are compared with a LSD test.

3. Formulations of the Essential Oil Extract

Formulations containing the essential oil extracts of the present invention can be prepared by known techniques to form emulsions, aerosols, sprays, or other liquid preparations, dusts, powders or solid preparations. These types of formulations can be prepared, for example, by combining with pesticide dispersible liquid carriers and/or dispersible solid carriers known in the art and optionally with carrier vehicle assistants, e.g., conventional pesticide surface-active agents, including emulsifying agents and/or dispersing agents. The choice of dispersing and emulsifying agents and the amount combined is determined by the nature of the formulation, the intended form of application of the formulation to a specific environment (e.g., plant, animal, soil, building), and the ability of the agent to facilitate the dispersion of the essential oil extract of the present invention while not significantly diminishing the pesticidal, acaricidal, insecticidal, and/or fungicidal activity of the essential oil extract.

Non-limiting examples of conventional carriers include liquid carriers, including aerosol propellants which are gaseous at normal temperatures and pressures, such as Freon; inert dispersible liquid diluent carriers, including inert organic solvents, such as aromatic hydrocarbons (e.g., benzene, toluene, xylene, alkyl naphthalenes), halogenated especially chlorinated, aromatic hydrocarbons (e.g., chloro-benzenes), cycloalkanes (e.g., cyclohexane), paraffins (e.g., petroleum or mineral oil fractions), chlorinated aliphatic hydrocarbons (e.g., methylene chloride, chloroethylenes), alcohols (e.g., methanol, ethanol, propanol, butanol, glycol), as well as ethers and esters thereof (e.g., glycol monomethyl ether), amines (e.g., ethanolamine), amides (e.g., dimethyl sormamide), sulfoxides (e.g., dimethyl sulfoxide), acetonitrile, ketones (e.g., acetone, methyl ethyl ketone, methyl isobutyl ketone, cyclohexanone), and/or water; as well as inert dispersible finely divided solid carriers such as ground natural minerals (e.g., kaolins, clays, vermiculite, alumina, silica, chalk, i.e., calcium carbonate, talc, attapulgite, montmorillonite, kieselguhr), and ground synthetic minerals (e.g., highly dispersed silicic acid, silicates).

Surface-active agents, i.e., conventional carrier vehicle assistants, that can be employed with the present invention include, without limitation, emulsifying agents, such as non-ionic and/or anionic emulsifying agents (e.g., polyethylene oxide esters of fatty acids, polyethylene oxide ethers of fatty alcohols, alkyl sulfates, alkyl sulfonates, aryl sulfonates, albumin hydrolyzates, and especially alkyl arylpolyglycol ethers, magnesium stearate, sodium oleate); and/or dispersing agents such as lignin, sulfite waste liquors, methyl cellulose.

Emulsifiers that can be used to solubilize the essential oil extracts of the present invention in water include blends of anionic and non-ionic emulsifiers. Examples of commercial anionic emulsifiers that can be used include, but are not limited to: Rhodacal™ DS-10, Cafax™ DB-45, Stepanol™ DEA, Aerosol™ OT-75, Rhodacal™ A246L, Rhodafac™ RE-610, and Rhodapex™ CO-436, Rhodacal™ CA, Stepanol™ WAC. Examples of commercial non-ionic emulsifiers that can be used include, but are not limited to: Igepal™ CO-887, Macol™ NP-9.5, Igepal™ CO-430, Rhodasurf™ ON-870, Alkamuls™ EL-719, Alkamuls™ EL-620, Alkamide™ L9DE, Span™ 80, Tween™ 80, Alkamuls™ PSMO-5, Atlas™ G1086, and Tween™ 20, Igepal™ CA-630, Toximul™ R, Toximul™ S, Polystep™ A7 and Polystep™ B1.

If desired, colourants such as inorganic pigments, for example, iron oxide, titanium oxide, and Prussian Blue, and organic dyestuffs, such as alizarin dyestuffs, azo dyestuffs or metal phthalocyanine dyestuffs, and trace elements, such as salts of iron, manganeses, boron, copper, cobalt, molybdenum and zinc may be used.

Spreader and sticking agents, such as carboxymethyl cellulose, natural and synthetic polymers (e.g., gum arabic, polyvinyl alcohol, and polyvinyl acetate), can also be used in the formulations. Examples of commercial spreaders and sticking agents which can be used in the formulations include, but are not limited to, Schercoat™ P110, Pemulen™ TR2, and Carbose™ 514 H, Umbrella™, Toximul™858 and Latron™CS-7.

Time-release formulations are also contemplated by the present invention. For example, formulations which have been encapsulated and/or pelletized.

In one embodiment, the formulation is a sprayable ready-to-use (RTU) formulation suitable, for example, for delivery by fogging, aerosol spraying, and pump spraying methods for application in a variety of environments.

In a further embodiment, the formulation can also be prepared as an emulsifiable concentrate (EC) which can be diluted before use. In one embodiment, the emulsifiable concentrate comprises between 5% to 50% (by volume) Chenopodium-derived essential oil extract in combination with a suitable emulsifier, carrier, and spreader and/or sticking agent to enable application of the formulation to a specific environment. In another embodiment, the emulsifiable concentrate comprises between 10% to 25% (by volume) Chenopodium-derived essential oil extract in combination with a suitable emulsifier, carrier, and spreader and/or sticking agent to enable application of the formulation to a specific environment. In a further embodiment, the emulsifiable concentrate comprises between 10% to 25% (by volume) Chenopodium-derived essential oil extract in combination with between 1% to 15% (by volume) of a suitable emulsifier, and between 50% to 70% (by volume) of a suitable carrier or solvent to enable application of the formulation to a specific environment. In another embodiment, the emulsifiable concentrate may also comprise 2% to 20% (by volume) of a suitable spreader and/or sticking agent. The person skilled in the art, however, will understand that these concentrations can be modified in accordance with particular needs so that the formulation is acaricidal, insecticidal, and/or fungicidal, but not phytotoxic.

Non-limiting examples of suitable emulsifiers that can be used in preparing emulsifiable concentrates of the present invention include: Rhodapex™ CO-436, Rhodapex™ CO-433, Igepal™ CO-430, Igepal™ CA-630, Igepal™ CO-887, Isopropanol, canola oil, Alkamuls™ EL-719, Rhodacal™ DS-10, Macol™ NP-9.5, Tergitol™ TMN-3, Tergitol™ TMN-6, Tergitol™ TMN-10, Morwet™ D425, and Tween™ 80.

Suitable carriers or solvents that may be used include, but are not limited to, Isopar™ M, THFA™, ethyl lactate, butyl lactate, Soygold™ 1000, M-Pyrol, Propylene glycol, Agsolex™ 12, Agsolex™ BLO, Light mineral oil, Polysolve™ TPM, and Finsolv™ TN.

Examples of suitable spreaders and/or sticking agents include, but are not limited to, Latex emulsion, Umbrella™, Adsee™ 775, Witconol™ 14, Toximul™ 858, Latron™ B-1956, Latron™ CS-7, Latron™ AG-44M, T-Mulz™ AO-2, T-Mulz™ 1204, Silwet™ L-774.

Formulations containing the essential oil extracts of the present invention can be prepared by known techniques to enable application to specific environments.

Plant Formulations

The formulation can be prepared for application to plants and plant environments, for example, household/domestic plants, greenhouse plants, agricultural plants, and horticultural plants. In one embodiment, the formulation contains a final concentration of between 0.125% to 10% (by volume) of the Chenopodium-derived essential oil extract in combination with a suitable emulsifier, carrier, and spreader and/or sticking agent to enable sprayable application to a plant. In another embodiment, the formulation contains between 0.25% to 5% (by volume) of the Chenopodium-derived essential oil extract in combination with a suitable carrier, emulsifier, and spreader and/or sticking agent, to enable sprayable application to a plant.

Fumigant Formulations for Closed and/or Open Environments

The formulation can also be prepared for application as a fumigant for both outdoor as well as indoor application, for example in closed environments, such as greenhouses, animal barns or sheds, human domiciles, and other buildings. Persons of skill in the art will appreciate the various methods for preparing such fumigants, for example, as fogging concentrates and smoke generators. A fogging concentrate is generally a liquid formulation for application through a fogging machine to create a fine mist that can be distributed throughout a closed and/or open environment. Such fogging concentrates can be prepared using known techniques to enable application through a fogging machine. For example, the formulation may have the following general composition:

| Ingredient | % |
|---|---|
| Chenopodium extract (active ingredient) | 1-15 |
| Spreader/sticker | 1-5 |
| Organic solvent | To 100% |

Smoke generators, which are generally a powder formulation which is burned to create a smoke fumigant. Such smoke generators can also be prepared using known techniques. For example, the formulation may have the following general composition:

| Ingredient | % |
|---|---|
| Chenopodium extract (active ingredient) | 1-10 |
| Absorbent silica | 3-5 |
| Pyrotechnic ingredients | 3-10 |
| Free flow aid | 1-5 |
| Filler | To 100% |

Animal Formulations

The formulation can be prepared in a form suitable for application to animals and animal environments, such as barns or sheds. In one embodiment, the formulation is prepared in a form suitable for topical application on an animal for controlling insects, acari, pests and fungi. Persons of skill in the art will appreciate the various methods for preparing such topical formulations, for example as powders or sprayable formulations. In one embodiment, the topical formulation is an aerosol and may have the following general composition:

| Ingredient | % |
|---|---|
| Chenopodium extract (active ingredient) | 1-5 |
| Emollient (coat conditioner) | 2-5 |
| Stabilizer | 0.1-1 |
| Preservative | 0.1-0.5 |
| Organic solvent | To 100% |

In another embodiment, the formulation is a water-based pump spray formulation having the following general composition:

| Ingredient | % |
|---|---|
| Chenopodium extract (active ingredient) | 1-10 |
| Emulsifier(s) | 2-10 |
| Coat conditioner | 2-5 |
| Stabilizer | 0.1-1 |
| Preservative | 0.1-1 |
| Antioxidant | 0.1-0.5 |
| Water | To 100% |

In a further embodiment, the formulation is an aerosol formulation for application to an animal environment, such as a barn or shed, having the following general composition:

| Ingredient | % |
|---|---|
| Chenopodium extract (active ingredient) | 3-10 |
| Stabilizer | 0.1-1 |
| Preservative | 0.1-0.5 |
| Organic solvent | To 100% |

Human Formulations

The formulation can be prepared in a form suitable for topical application on humans, for example as a repellent. Persons of skill in the art will appreciate the various methods for preparing such topical formulations, for example as lotions or sprayable formulations. In one embodiment, the formulation is a solvent-based sprayable formulation having the following general composition:

| Ingredient | % |
|---|---|
| Chenopodium extract (active ingredient) | 1-5 |
| Emollient (skin conditioner) | 2-5 |
| Stabilizer | 0.1-1 |
| Organic solvent | To 100% |

In another embodiment, the formulation is a water-based sprayable formulation having the following general composition:

| Ingredient | % |
|---|---|
| Chenopodium extract (active ingredient) | 1-5 |
| Emulsifier(s) | 1-5 |
| Skin conditioner | 2-5 |
| Stabilizer | 0.1-1 |
| Preservative | 0.1-1 |
| Antioxidant | 0.1-0.5 |
| Water | To 100% |

In a further embodiment, the formulation is a topical lotion formulation having the following general composition:

| Ingredient | % |
|---|---|
| Chenopodium extract (active ingredient) | 1-10 |
| Emulsifier(s) | 1-10 |
| Skin conditioner | 2-10 |
| Thickener(s) | 0.2-5 |
| Stabilizer | 0.1-1 |
| Preservative | 0.1-1 |
| Antioxidant | 0.1-0.5 |
| Water | To 100% |

Soil Formulations

In a further embodiment, the formulation of the present invention can be prepared as a microemulsion. Microemulsions are low-viscosity, optically transparent dispersions of two immiscible liquids which are stabilized by at least one ionic or nonionic surfactant. In the case of microemulsions, the particle diameters are in the range from about 5-100 nm suspended in a continuous phase. The interfacial tension between the two phases is extremely low. The viscosity of many microemulsions of the oil and water type (O/W) is comparable with that of water.

In contrast to microemulsions, “macroemulsions” have high viscosities and their particle diameter is in the range from about 10 to 100 micrometers. Macroemulsions are milky white in color and, upon heating, tend toward phase separation or toward sedimentation of the dispersed substances. It is commonly believed that pesticidal microemulsions can provide superior efficacy relative to macroemulsion formulas having the same levels of active ingredients. It is believed that the small size of the emulsion droplets may allow for better transport of the pesticide through cell membranes (plant and insect) thereby resulting in enhanced efficacy. Microemulsions are considered to be infinitely stable, thereby providing improved stability over traditional macroemulsion systems. Accordingly, microemulsion formulations of the present invention may be particularly suitable for certain applications, for example soil delivery.

Microemulsion formulations of the present invention can be prepared using various known methods known in the art. In one embodiment, the essential oil extract of the present invention can be combined with a combination of emulsifiers to create a microemulsion. For example, a microemulsion can be prepared in a three phase process.

Phase 1: Selection of Emulsifiers

A selection of potential primary emulsifiers is tested for miscibility with the active ingredient. Solubility is evaluated by visually assessing for a clear stable solution. Each pesticide/emulsifier mixture is mixed with the required volume of water and the quality of emulsion/microemulsion produced is recorded. Emulsifiers producing the best emulsions are taken through into phase 2. Emulsion/microemulsion quality is determined by visual assessment. Transparent microemulsions are the best, white emulsions with a bluish hue are second, plain white emulsions are next and any emulsion showing phase separation is last.

Phase 2: Selection of Secondary and Tertiary Emulsifiers

Upon completion of phase 1, the most promising primary emulsifier systems are blended with other emulsifiers in various ratios until an emulsifier blend is identified which provides the desired physical properties. The optimized system generally consists of one main emulsifier and two or more co-emulsifiers/co-solvents. These co-emulsifiers/co-solvents tend to broaden the thermal phase stability range of the microemulsion system. Ternary phase diagrams are used in this phase of development to assist in the selection of emulsifier ratios. The best formulation at this stage is often one containing one anionic emulsifier and two non-ionics of different types. The criteria for assessing the microemulsion properties are again physical appearance as a function of time and temperature.

Phase 3: Optimization of Emulsifier Levels

Having established the best emulsifier combination, the final phase is optimizing the levels and ratios of each emulsifier component in the system.

In one embodiment, the microemulsion comprises between 30% to 50% (by volume) Chenopodium-derived essential oil extract, between 0.5% to 25% (by volume) of a suitable emulsifier, and between 10% to 50% water. Non-limiting examples of emulsifiers that can be used in preparing microemulsions of the present invention include: Igepal™ CA-630, Rhodasurf™ ON-870, Alkamuls™ EL-719, Tween™ 80, Alkamuls™ PSMO-5, Rhodapex™ CO-436, Rhodafac™ RE-610, Rhodacal™ CA, Ammonyx™ CO, Aeros™ OT-S, Ammonyx™ LO, Stepanol™ WAC, Soprophor™ BSU, and Rhodaca™1 IPAM.

It is contemplated that the formulations of the essential oil extract can be combined with a controlled release delivery system in order to time-release the bioactive agents. Such controlled release delivery systems include methods, known in the art, of encapsulation, dissolution, or incorporation of the active ingredient. It is further contemplated that the formulations of the present invention can be prepared in combination with nutrients (fertilizers) or herbicides. Thus the skilled artisan will appreciate that the instant invention may be further formulated to provide various dissolution rates and/or be prepared in combination with nutrients (fertilizers) or herbicides.

4. Use of Essential Oil Extract Formulations

The essential oil extracts of the present invention can be used for controlling pests by applying a pesticidally effective amount of the essential oil extract and/or formulation of the present invention to the locus to be protected. The essential oil extract formulations can be applied in a suitable manner known in the art such as, for example, spraying, atomizing, vaporizing, scattering, dusting, watering, squirting, sprinkling, pouring, fumigating, and the like. Loci, for the application of essential oil extracts or formulations thereof include, but are not limited to, agricultural, horticultural, forest, plantation, orchard, nursery, organically grown crops, turfgrass and urban environments. Specifically, the essential oil extracts or formulations thereof may be applied to soil or plants/trees at one or several sites including the leaves, petioles, stems, seeds, roots, flower, cones, bark, wood or tubers. It is further contemplated that the essential oil extract may be formulated for seed treatment either as a pre-treatment for storage or sowing. For example, the seed may form part of a pelleted composition or, alternatively, may be soaked, sprayed, dusted or fumigated with a formulation of the present invention. The essential oil extract, and formulations thereof, can be applied to the surface of the soil to control soil surface or soil-inhabiting pests. The essential oil extract of the present invention can also be used as part of an Organic Production system and Integrated Pest Management program. For example, in conjunction with augmentation of beneficial insects and mites. It is further contemplated that the essential oil extract, and formulations thereof, can be applied to ‘fertigation’ i.e. fertilization via the irrigation system of plants in greenhouses. For example, an essential oil microemulsion can be added to the water in small concentrations (0.1-0.5% AI) and as water is being irrigated to individual plants, the latter would be treated for soil-inhibating pests (insects and disease pathogens).

5. Effect of the Essential Oil Extract or Formulations on Beneficial Insects and Mites

Natural enemies of phytophagous pests include both predators and parasitoid. Predators are generally as large, or larger than the prey they feed on. They are quite capable of moving around to search out their food, and they usually consume many pests during their lifetime. Parasitoids, or parasitic insects, are smaller than their prey. One or more parasitoids grow and develop in or on a single host. The host is slowly destroyed as the parasitic larva(e) feed and mature. Such beneficial insects and mites can help prevent or delay the development of pesticide resistance by reducing the number of pesticides required to control a pest. They will also feed on the resistant pests that survive a pesticide application.

Integrated pest management (IPM) programs take advantage of the biological pest control provided by beneficial insects and mites by conserving or augmenting natural enemies. When chemical controls are necessary in an IPM program, pesticides recommended are those that have minimal impact on naturally occurring beneficials.

Essential oil extracts of the present invention, and formulations thereof, may be tested for their effect on beneficial insects and mites, i.e., predators and parasitoids, by means of standardized IOBC (International Organization for Biologicial Control) testing methods as illustrated in Example XIV for integration into IPM programs.

The invention now being generally described, it will be more readily understood by references to the following examples, which are included for purposes of illustration only and are not intended to limit the invention unless so stated.

### EXAMPLES

### Example I

### Phytochemical Profile of an Essential Oil Extract Derived from Chenopodium ambrosioides

Whole plants of C. ambrosioides were harvested. Plant material used for extraction purposes comprised the whole plant above root. Essential oil extracts were extracted from the plant material by steam distillation, i.e., distillation in water (DW) and/or direct steam distillation (DSD).

Distillation in water was carried out in a 380 L distillator with a capacity for processing ca. 20 kg of plant material. During the process of DW, plant material was completely immersed in an appropriate volume of water which was then brought to a boil by the application of heat with a steam coil located at the base of the still body. In DSD, the plant material was supported within the still body and packed uniformly and loosely to provide for the smooth passage of steam through it. Steam was produced by an external generator and allowed to diffuse through the plant material from the bottom of the tank. The rate of entry of the steam was set at (300 ml/min). With both methods, the oil constituents are released from the plant material and with the water vapor are allowed to cool in a condenser to separate into two components, oil and water.

The essential oil extracts were analyzed by capillary gas chromatography (GC) equipped with a flame ionization detector (FID). GC was carried out using a Varian 6000 series Vista and peak areas were computed by a Varian DS 654 integrator. SPB-1 (30 m×0.25 mm φ, 0.25 μm) and Supelcowax (30 m×0.25 mm φ, 0.25 μm) fused silca columns were used. Compounds in the sample come off the column at different times in minutes (Rt's or Retention Times) and these are compared to known standards and the compounds can thus be identified. When GC-FID gave ambiguous identification of certain compounds, Mass Spectrometry (MS) was used to compare the mass spectra of the compounds with a database of known spectra.

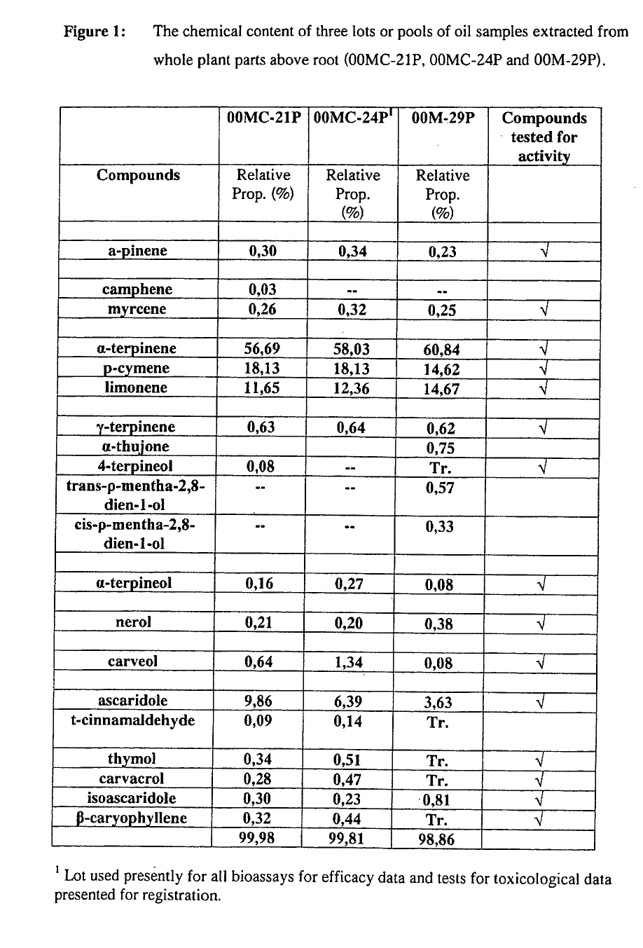

The relative amount of each component of the essential oil extracts was determined for different lots of a variety of C. ambroisiodes. Each lot represents pooled extractions taken from a crop within one harvest date. FIG. 1 shows the phytochemical profile of the essential oil extract taken from three different lots. Lot No. 00MC-21P indicates an ascaridole content of 9.86%; Lot No. 00MC-24P has an ascaridole content of 6.39% and 00MC-29P has an ascaridole content of 3.6%. The activity of the extract is not apparently affected by the variability in relative amount of ascaridole as results from bioassays with these lots suggest.

### Example II

### Determination of the Active Constituents of the Essential Oil Extract

Extensive testing was done in order to determine the active constituents of the essential oil extract. All compounds present in the oil were tested except for trans-ρ-mentha-2,8dien-1-ol and cis-ρ-mentha-2,8-dien-1-ol because they were unavailable. All compounds tested were obtained commercially (Sigma-Aldrich) except for ascaridole and iso-ascaridole that were isolated from a sample of our extract by Laboratoires LaSeve, Chicoutimi Qc.

Acaricidal Activity

Tests with the Two-Spotted Spider Mite (TSSM: Tetranychus urticae)

To test acaricidal activity, thirty adult female mites were placed on their dorsum with a camel hair brush on a double-sided sticking tape glued to a 9 cm Petri dish. Three dishes were prepared for each concentration of each compound tested and the control (e.g., water) for a total of 90 mites per treatment per treatment day.

One (1) ml of each preparation and of microfiltered water as control was added with a Gilson Pipetman™ P-1000 to the reservoir of the spray nozzle of a Potter Spray Tower mounted on a stand and connected to a pressure gauge set at 3 P.S.I. Petri dishes were weighed before and immediately after each application to calculate the amount of oil deposited (mg/cm²) with each sample tested. The entire procedure was followed three times to give a total number of 270 mites tested with each treatment.

Mite mortality was assessed 24 and 48 h after treatment. Mites that failed to respond to probing with a fine camel hair brush with movements of the legs, proboscis or abdomen were considered dead.

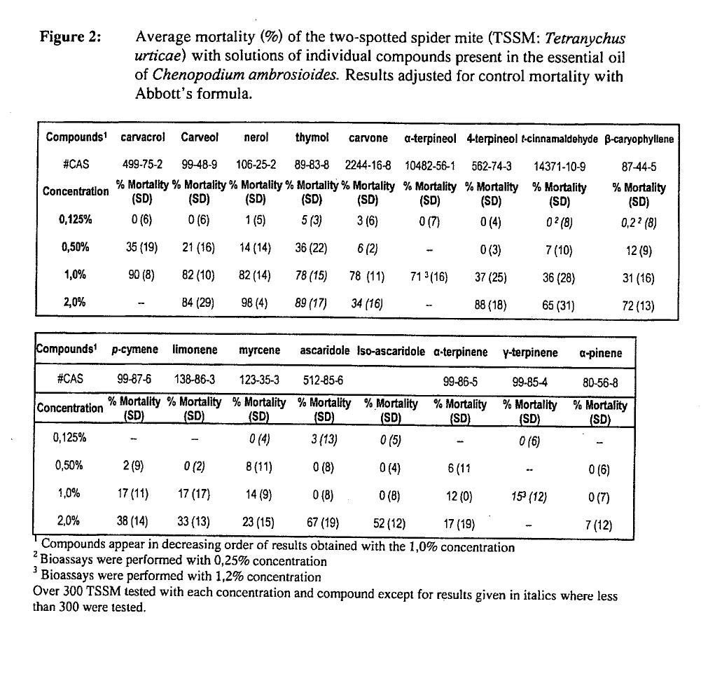

Individual compounds were tested at 0.125, 0.50, 1.0 and 2.0% concentrations with the two-spotted spider mite (TSSM: Tetranychus urticae). Results are illustrated in FIG. 2. Comparisons were made with mortality data obtained with the 1% concentration of each compound and it was observed that carvacrol is the most active compound (90% mortality of TSSM) followed by carveol (82% mortality), nerol (82% mortality), thymol (78% mortality), carvone (78% mortality) and α-terpineol (71% mortality). Other compounds gave less than 40% mortality. No mortality was recorded for ascaridole at 1% . Although 3% mortality was obtained with a solution of 0.125% ascaridole, we believe that this is an erroneous udependable result because too few individuals were tested (n=125) and the standard deviation is high (13), compared to the higher number of individuals tested at the higher concentrations of this compound (n=300 each at 0.5% and 1.0%) where no mortality was recorded.

The results obtained with individual compounds, do not indicate that the compounds present in large quantities in the oil, i.e α-terpinene, ρ-cymene, limonene, ascaridole, iso-ascaridole, have a great impact on the biological activity of the extract. Mortality obtained with each of these compounds tested at 1% concentration was 17% or less. Ascaridole and iso-ascaridole at 1% concentration had no effect on the spider mite (0% mortality).

Carvacrol, carveol, nerol, thymol and carvone on the other hand may have a much greater impact on the activity of the oil (>70% of TSSM at a 1% concentration) even though each of these compounds are present in relatively small quantities (<1%)

Insecticidal Activity

Tests with the Greenhouse Whitefly (GWF: Trialeurodes vaporariorum)

Tests were also done using compounds that had demonstrated the higher degree of activity, i.e. carvacrol, nerol and thymol with the greenhouse whitefly (Trialeurodes vaporariorum) our model bioassay for insecticidal effect.

Whitefly adults were glued to a black 5 cm×7,5 cm plastic card sprayed with Tangle-Trap® (Gempler's Co.) by placing cards directly in the greenhouse colony cage until at least 20 adults have alighted on each card. Cards were observed before spraying under the binocular scope to remove all dead and immobile whiteflies. Only active whiteflies were kept for the experiment. Four cards were used per treatment. Each card was sprayed at 6 psi with 300 μl of emulsion using a BADGER 100-F® (Omer DeSerres Co., Montréeal, Canada) paintbrush sprayer mounted on a frame at a distance of 14.5 cm from the spray nozzle in an exhaust chamber. Cards were weighed immediately before and after spraying to calculate the amount of active ingredient deposited in mg/cm². Cards were allowed to dry under the exhaust chamber and then placed sideways on a Styrofoam rack in a closed clear plastic container of 5 L with moistened foam on the bottom to keep humidity high (>90% R.H.). The plastic container was stored in a growth chamber at 24° C. and 16 L:8 D photoperiod. This procedure was repeated three times.

Mortality was evaluated 20 hours following treatment by gently probing the whitefly with a single-hair brush under the binocular microscope. Absence of movement (antennae, leg, wing) following probing was recorded as dead. Relative efficacy of the compounds were compared by transforming mortality data to arcsin√p and then subjecting to an ANOVA analysis using SAS® software (SAS Institute 1988).

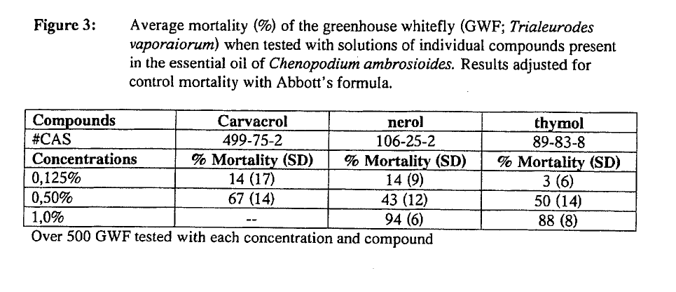

Results with the GWF, shown in FIG. 3, confirm the important biological activity of these three compounds.

### Example III

### Ready-To-Use Acaricidal Formulations

A ready-to-use (RTU) sprayable insecticidal formulation having as the active ingredient an extract of Chenopodium was prepared. In one embodiment, this formulation contains between 0.125% and 10% (by volume) of the essential oil extract, an emulsifier, a spreader and sticking agent, and a carrier.

Examples of basic RTU formulations are as follows.

| Ingredient | Amount (%) | Amount (%) | Amount (%) |
|---|---|---|---|
| Essential oil | 1.00 | 1.00 | 1.00 |
| extract | | | |
| Rodacal IPAM | 0.50 | 0.83 | 0.83 |
| Igepal CA-630 | — | 0.50 | — |
| Macol NP 9.5 | — | — | 0.50 |
| Water | 98.5 | 97.67 | 97.67 |

Examples of formulations with added polymers for added residual effect are as follows.

| Ingredient | Amount (%) | Amount (%) | Amount (%) |
|---|---|---|---|
| Essential oil extract | 1.00 | 1.00 | 1.00 |
| Rhodacal IPAM | 0.83 | 0.83 | 0.83 |
| Igepal CA-630 | 0.50 | 0.50 | 0.50 |
| Carboset 514H | 2.00 | — | — |
| Pemulen TR2 | — | 0.05 | — |
| Schercoat P110 | — | — | 5.00 |
| Propylene glycol | — | 2.00 | — |
| Water | 95.67 | 95.62 | 92.67 |

### Example IV

### Acaricidal Efficacy of the Essential Oil Extract (RTU Formulation)

Efficacy trials were conducted using a Ready-to-use (RTU) formulation of the present invention comprising: 1.00% Essential oil extract; 0.83% Rhodacal IPAM; 0.50% Igepal CA-630; 0.05% Pemulen TR2; 2.00% Propylene glycol; and 95.62% Water.

Thirty adult female mites were placed on their dorsum with a camel hair brush on a double-sided adhesive tape glued to a 9 cm Petri dish. Three dishes were prepared for each concentration of each formulations or products tested and the control, (e.g. water), for a total of 90 mites per treatment per treatment day.

One (1) ml of each preparation and of microfiltered water as control was added with a Gilson Pipetman™P-1000 to the reservoir of the spray nozzle of a Potter Spray Tower mounted on a stand and connected to a pressure gauge set at 3 P.S.I. Petri dishes were weighed before and immediately after each application to calculate the amount of oil deposited (mg/cm²) with each sample tested.

The ready-to-use formulation was tested according to the method mentioned above to identify the minimum concentration needed for the desired mortality (>95%) at different concentrations (00.125, 0.25, 0.5, 0.75, and 1%) in order to compare the relative efficacy of this RTU formulation and other acaricidal products (synthetic and natural) presently on the market.

The entire procedure was followed three times to give a total number of 270 mites tested with each treatment.

Mite mortality was assessed 24 and 48 h after treatment. Mites that failed to respond to probing with a fine camel hair brush with movements of the legs, proboscis or abdomen were considered dead. In order to obtain LC₅₀ values (Lethal Concentration in mg/cm² is the amount of product needed to kill 50% of the test organism; therefore the lower the LC₅₀ value the more toxic the product) results of the 48 h counts were subjected to Probit analysis using POLO computer program (LeOra Software, 1987). Mortalities were entered with corresponding weighed dose (mg/cm²) to take into consideration variability in the application rate.

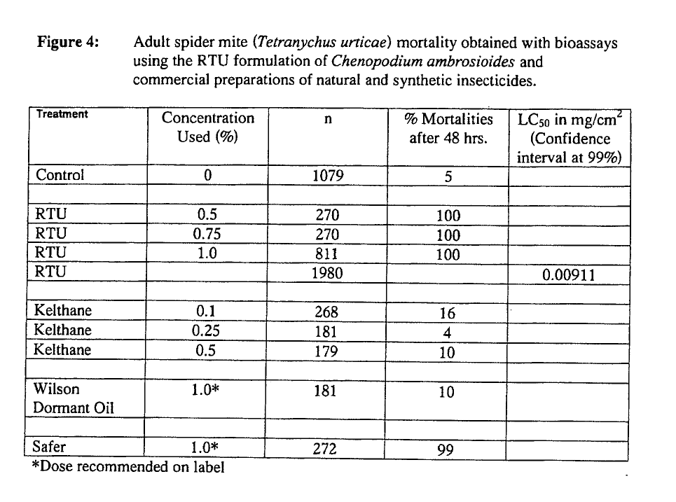

The results obtained with these bioassays are shown in FIG. 4.

Although the toxicity tests presented herein were performed with female mites, it will be clear to a person skilled in the art that those results show that the mortality that would have been observed for male mites would have been the same if not higher knowing that male mites are smaller than females.

### Example V

### Effect on the Egg and Nymphal Stages of the Spider Mite (RTU Formulation)

The RTU formulation (comprising: 1.00% Essential oil extract; 0.83% Rhodacal IPAM; 0.50% Igepal CA-630; 0.05% Pemulen TR2; 2.00% Propylene glycol; and 95.62% Water) was also tested on the egg and the nymphal stages of the spider mite. The ovicidal effect was determined with eggs of the twospotted spider mite following treatment with concentrations of the RTU formulation. Adult female T. urticae are transferred to 2 cm diameter leaf disks cut out of lima bean leaves and left for four hours for oviposition. When at least 20 eggs/disk are laid, adult mites are then removed. Leaf disks are moist and then sprayed and Petri dishes are weighed before and after treatment and stored after treatment. Egg hatch is assessed daily and for 10 days following treatment by counting the number of eggs remaining on the leaf disks and the number of live and dead nymphs present. Percent egg hatch is determined with live nymphs only. The nymphs are considered dead if no movement is observed after repeated gentle probing with a single-hair brush.

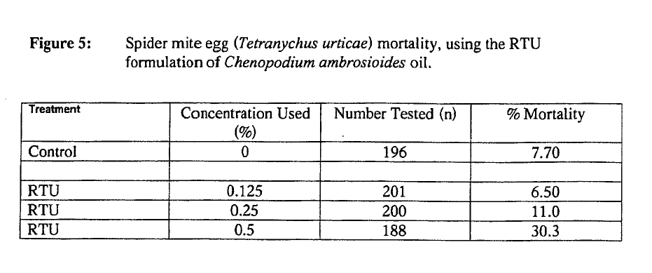

Results of the test on the egg stage (FIG. 5) indicate that the RTU formulation has some effect on the eggs with 30% mortality using a 0.5% solution of the oil. It is expected that a higher concentration of the oil should show greater efficacy on eggs.

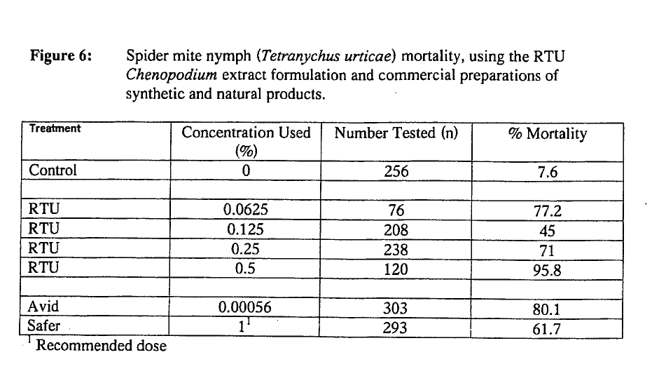

Similarly to the effect of the RTU formulation on the nymphal stage, even at the 0.5% concentration, the RTU gave higher results (95.8%) than the existing commercial preparations of either Avid (80.1%) or Safer Soap (61.7%) (FIG. 6).

### Example VI

### Residual Effect of the RTU Formulations of the Present Invention and Comparison Thereof with Commercially Available Acaricidal Products

The residual effect of the RTU formulation (comprising: 1.00% Essential oil extract; 0.83% Rhodacal IPAM; 0.50% Igepal CA-630; 0.05% Pemulen TR2; 2.00% Propylene glycol; and 95.62% Water) was also tested with the spider mite and compared to natural and synthetic products already on the market, (i.e. Kelthane™, Avid™, Safer's™ Soap and Wilson's dormant oil). The procedure for this test involved the preparation of vials containing a nutrient solution in which individual faba bean leaves were placed. Eighteen leaves were prepared for each concentration tested and each were sprayed with the indicated concentration until run-off and allowed to dry. Ten spider mites were placed on nine of the leaves one hour after spraying and ten were placed on the other nine leaves one day following treatment. Mortality was observed 24 and 48 hr following mite introduction on the leaves. The entire procedure was repeated three times.

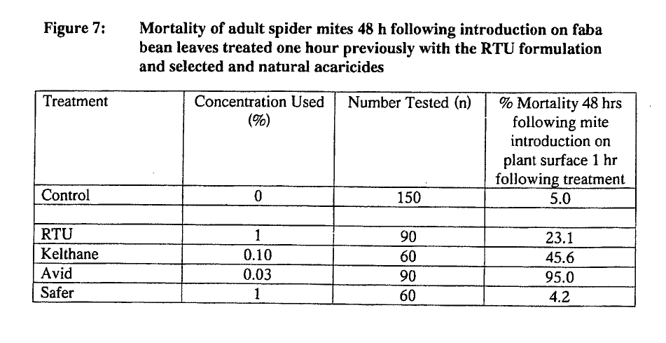

The results of the residual effect of the different products when the mite is introduced on the plant one hour following treatment are shown in FIG. 7. These results indicate that there is a residual effect of the RTU and that this effect is greater than in the Safer product. However, it is inferior to the residual effect of synthetic products such as Kelthane and Avid.

These results show the RTU formulation's very low persistence in the environment (about 23 mortality of spider mites when the pest is introduced on the plant one hour after treatment with the product). The RTU formulation is therefore compatible with the recommendations of the Integrated Pest Management program which supports control methods that do not harm natural enemy populations and permit rapid re-entry of workers to the tested area and uninterrupted periods of harvest while assuring safety to workers and consumers.

### Example VII

### Acaricidal Activity of the Extracts on Other Acari (RTU Formulation)

To confirm the efficacy of the formulations of the present invention on plant infesting acari in general, certain bioassays were performed on another plant infesting mite, the European red mite, Panonychus ulmi, a mite which shows a close taxonomical relationship with T. Urticae.

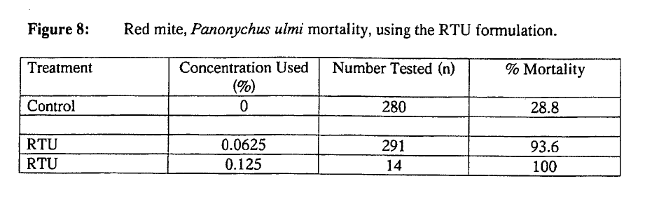

The RTU formulation (comprising: 1.00% Essential oil extract; 0.83% Rhodacal IPAM; 0.50% Igepal CA-630; 0.05% Pemulen TR2; 2.00% Propylene glycol; and 95.62% Water) was thus tested on the red mite Panonychus ulmi, a pest of apple orchards, following the same protocol described for contact efficacy on adult spider mites in order to confirm its broad effect as an acaricide. The results confirm the effectiveness of the essential oil extract as a contact acaricide (FIG. 8) which is not exclusively active on T. Urticae.

### Example VIII

### Insecticidal Efficacy of the Essential Oil Extract (RTU Formulation)

Similar efficacy tests were also performed on several insect species that are serious pests of cultivated plants. The species tested were the greenhouse whitefly, Trialeurodes vaporariorum; the Western flower thrips, Frankliniella occidentalis; the green peach aphid, Myzus persicae; and the silverleaf whitefly, Bermisia argentifolii following the same protocol described in Example XI (C) below.

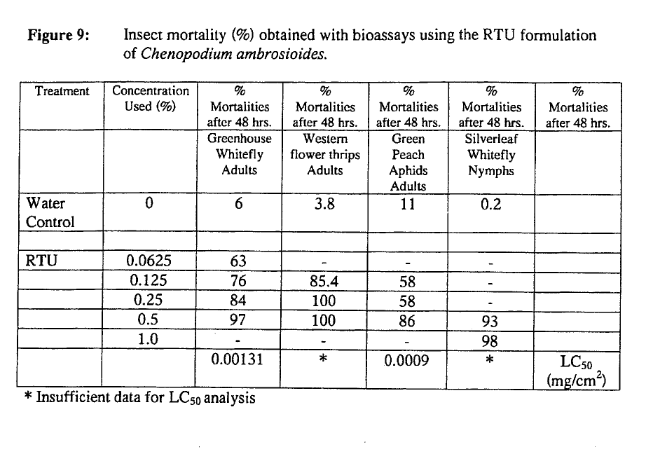

Results presented in FIG. 9 indicate that the RTU product (comprising: 1.00% Essential oil extract; 0.83% Rhodacal IPAM; 0.50% Igepal CA-630; 0.05% Pemulen TR2; 2.00% Propylene glycol; and 95.62% Water) is toxic to all organisms tested. LC₅₀ could be calculated for the greenhouse whitefly and the green peach aphid and results (LC₅₀ of 0.00131 mg/ cm² and 0.0009 mg/cm respectively) show that the product is as or more effective to these insects as the spider mite.

### Example IX

### Emulsifiable Concentrate Formulation

An emulsifiable concentrate formulation with an extract of Chenopodium ambrosioides was also prepared. The concentrate contains between 10 to 25% essential oil extract, emulsifiers, a spreader/sticker, and a carrier.

Examples of emulsifiable concentrate formulations are as follows.

| | Amount | Amount | Amount | Amount | Amount | Amount |
|---|---|---|---|---|---|---|
| Ingredient | (%) | (%) | (%) | (%) | (%) | (%) |
| Essential | 25 | 25 | 25 | 25 | 25 | 25 |
| oil extract | | | | | | |
| Rhodopex | 5 | 2.5 | — | — | 1.25 | — |
| CO-436 | | | | | | |
| Rhodopex | — | — | — | — | — | — |
| CO-433 | | | | | | |
| Igepal CO- | — | 2.5 | — | — | 1.25 | 2.5 |
| 430 | | | | | | |
| Igepal CA- | — | — | 5 | 2.5 | — | — |
| 630 | | | | | | |
| Igepal CO- | — | — | — | 2.5 | — | — |
| 887 | | | | | | |
| Isopropanol | — | — | 10 | — | — | — |
| Isopar M | — | — | 60 | 70 | — | — |
| Macol NP | — | — | — | — | — | 2.5 |
| 95 | | | | | | |
| THFA | 70 | 70 | — | — | 72.5 | 70 |

### Example X

### Acaricidal Efficacy of the Essential Oil Extract (Emulsifiable Concentrate Formulation)

Contact and residual bioassays were conducted in the laboratory to test the efficacy of the essential oil extract of the present invention. A 25% emulsifiable concentrate (EC25%) formulation of oil was tested against the adult and eggs of the twospotted spider mite and the European red mite. The EC25% formulation tested comprised: 25% Essential oil extract; 2.5% Rhodopex CO-436; 2.5% Igepal CO-430; and 70% THFA.

The twospotted spider mite was reared on Lima bean plants (Phaseolus sp.) and the European red mite on apple leaves cv McIntosh (Malus domestica Borkhausen).

Contact Efficacy with the Adult Stage

The methodology used for adults was the same for both species. Twospotted spider mite adults were treated with four concentrations of oil of a North American herbaceous plant (0.125, 0.25, 0.5 and 1.0% active ingredient (AI) EC25%; Urgel Delisle et Associes, Saint-Charles-sur-Richelieu, QC, Canada), neem oil (Neem Rose Defense® EC 90%; Green Light, San Antonio Tex, USA) at 0.7% AI, insecticidal soap (Safer's Trounce® EC 20% potassium salts of fatty acids with 0.2% pyrethrins; Safer Ltd. Scaborough, ON, Canada) at 1% AI and a water control. European red mite. adults were treated with five concentrations (0.0312, 0.0625, 0.125, 0.25 and 0.5%) of EC25%, abamectin (Avid® EC1.9%; Novartis, Greensboro, N.C., USA) at 0.006% AI and a water control.

Twenty-five mature female mites were deposited dorsally on a 1 cm² piece of double-coated tape glued on a glass microscope slide. For each treatment period, four slides were prepared for each treatment or acaricide application as defined above. Solutions for each treatment were prepared on the treatment day and each slide was sprayed at a pressure of 0.42 kg/cm² under an exhaust chamber with 250 μl of solution using a Badger 100-F® paint brush sprayer (Badger Air-Brush Co., Franklin Park, Ill., USA) mounted on a frame at a distance of 15 cm from the slide. The slides were weighed immediately before and after spraying to calculate the amount of active ingredient deposited per surface area (mg/cm²); this quantity varied less than 15% between slides. After spraying, the slides were placed on a styrofoam rack in a closed clear plastic container with a wet foam at the bottom to keep moisture high (90% R.H.). The container was stored in a growth chamber at 24° C. and 16 L:8 D photoperiod. This experimental procedure was repeated on three consecutive days in a complete block design where treatment period was considered a block.

Mortality was assessed under a binocular microscope 48 (twospotted spider mite) and 24 hours (European red mite) following treatment. Because European red mite mortality in the control group at 48 hours was high, it was judged to be inadequate for statistical evaluation. Mites were considered dead if movement was imperceptible after repeated gentle probing with a single-hair brush. Data were transformed by arcsin√p and subjected to an ANOVA statistical analysis using SAS® software (SAS Institute, 1988). The LC₅₀ and LC₉₀ (in mg/cm² of AI) of EC25% were calculated with PROBIT analysis using POLO-PC® software (LeOra Software, 1987).

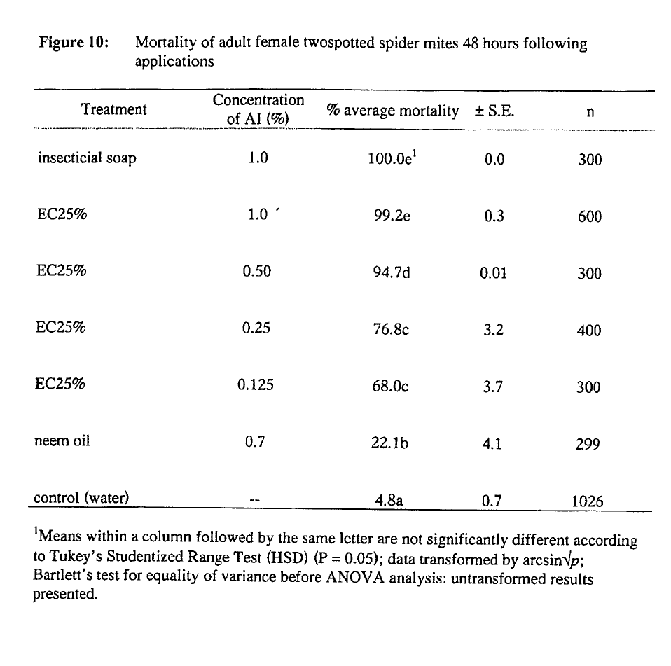

EC25% at 1% concentration and insecticial soap at 1% were most effective at controlling the adult twospotted spider mites causing 99.2 and 100% mortality respectively (FIG. 10). At 0.5, 0.25 and 0.125% EC25% resulted in 94.7, 76.8 and 68% mortality respectively. The least effective treatment was neem oil, which at the recommended dose caused only 22.1% mortality. The LC₅₀ and LC₉₀ of EC25% for the twospotted spider mite were 0.009 mg/cm² (99% confidence interval 0.0082-0.0099 mg/cm²) and 0.0292 mg/cm² (99% confidence interval 0.0268-0.0321 mg/cm²) respectively (significant at P=0.01). In comparison, the LC₅₀ of insecticial soap had been determined by the manufacturer to be 0.016 mg/cm².

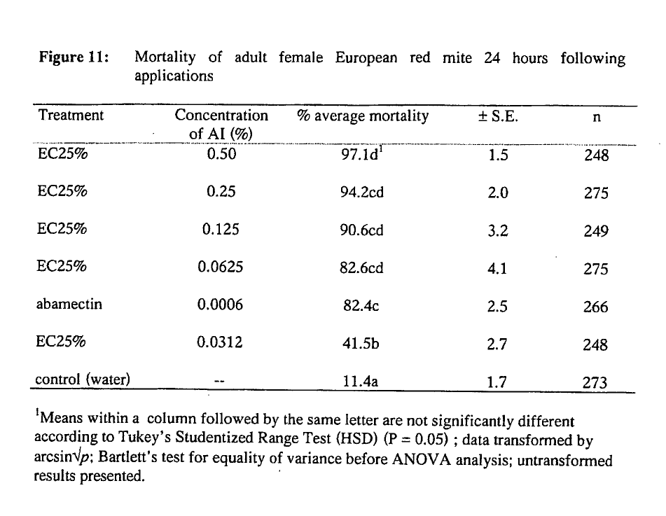

At 0.5% concentration, EC25% was significantly more toxic (97.1% mortality) to P. ulmi adults than abamectin (82.4%) (FIG. 11). Treatments with EC25% at concentrations ranging from 0.0625 to 0.25% gave statistically the same control level as abamectin. The LC₅₀ and LC₉₀ of EC25% for the red spider mite were 0.0029 mg/cm² (99% confidence interval 0.0019-0.0038 mg/cm²) and 0.014 mg/cm² (99% confidence interval 0.0108-0.0203 mg/cm²). EC25% gave <80% control of the adult stage of the two mites species at low doses.

Ovicidal Activity

The ovicidal effect of the following products was determined with eggs of the twospotted spider mite and the European red mite: six concentrations of EC25% (0.0625, 0.125, 0.25, 0.5, 1 and 2%), neem oil at 0.7% AI, insecticidal soap at 1% AI and abamectin at 0.006% and a water control. Twenty adult female T. urticae were transferred to 2 cm diameter leaf disks cut out of lima bean leaves and left for four hours for oviposition. Female P. ulmi were left for 24 hours to lay their eggs on 2 cm diameter leaf disks of apple leaves. When at least 20 eggs/disk were laid, adult mites were then removed with a soft brush Leaf disks were kept on moist soft cotton swabs placed in small (4 cm diameter) plastic Petri dishes. Three leaf disks were prepared for each treatment or acaricide application. Leaf disks were sprayed and Petri dishes were weighed before treatment and stored after treatment as for the slides used in the bioassay with adults. This experimental procedure was repeated on three consecutive days in a complete block design where treatment period was considered a block.

Egg hatch was assessed daily and for 10 days following treatment by counting the number of eggs remaining on the leaf disks and the number of live and dead nymphs present. Percent egg hatch was determined with live nymphs only. The nymphs were considered dead if no movement was observed after repeated gentle probing with a single-hair brush. All nymphs (alive and dead) were removed daily from the leaf disks. Percent egg hatch (number of nymphs/total number of eggs on leaf disk×100) were transformed with arcsin √ [symbol] and subjected to an ANOVA statistical analysis using SAS® software (SAS Institute, 1988).

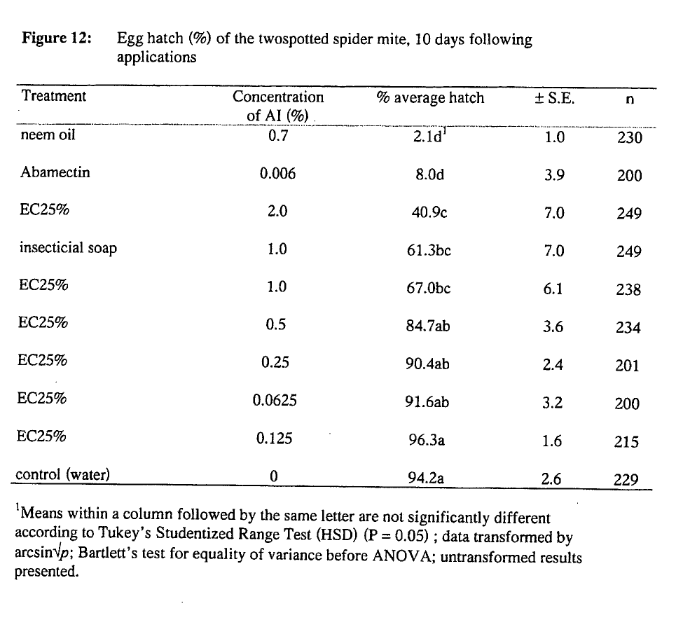

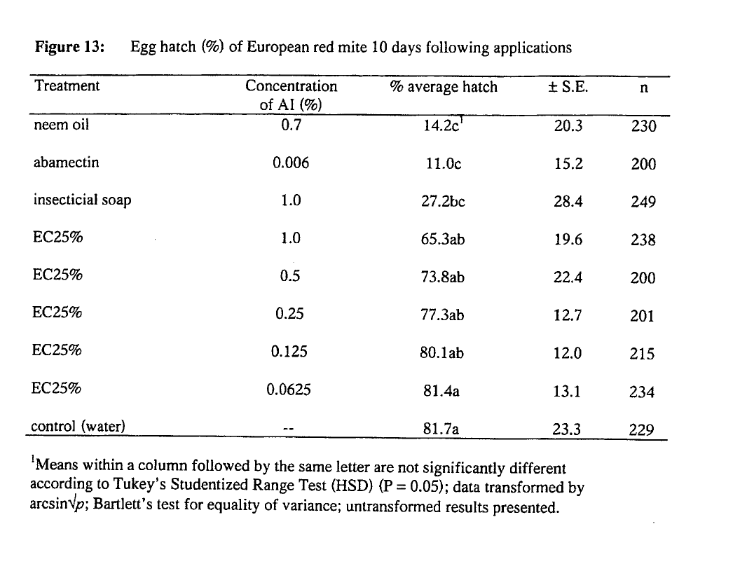

Egg hatch for the twospotted spider mite was significantly reduced by abamectin (8.0% egg hatch) and neem oil (2.1%) (FIG. 12). Egg hatch was reduced to 67 and 40% with 1.0 and 2.0% concentrations of EC25% respectively and to 61.3% with insecticial soap. Egg hatch for the European Red mite was significantly reduced compared to the control treatment with the recommended doses of insecticial soap (27.2% egg hatch), abamectin (11.0%) and neem oil (14.2%) (FIG. 13).

Residual Bioassay with Adult Twospotted Spider Mites

Leaf discs measuring 2 cm in diameter of bean leaves were sprayed on both sides with a VEGA 2000 sprayer (Thayer & Chandler Co., Lake Bluff, Ill., USA) at 0.42 kg/cm² to runoff with 6.25 ml of each the following solutions: 2, 4, 8, and 16% of 99B-245, the recommended dose of dicofol (Kelthane® 35WP, Rohm and Haas Co., Philadelphia, Pa., USA) at 0.037% AI and a water control. Each treatment consisted of eight discs. One hour after treatment, 10 spider mites were transferred to each disc. Mortality was evaluated 48 hours following transfer of mites to the leaf discs. The procedure was repeated three times on three subsequent days.

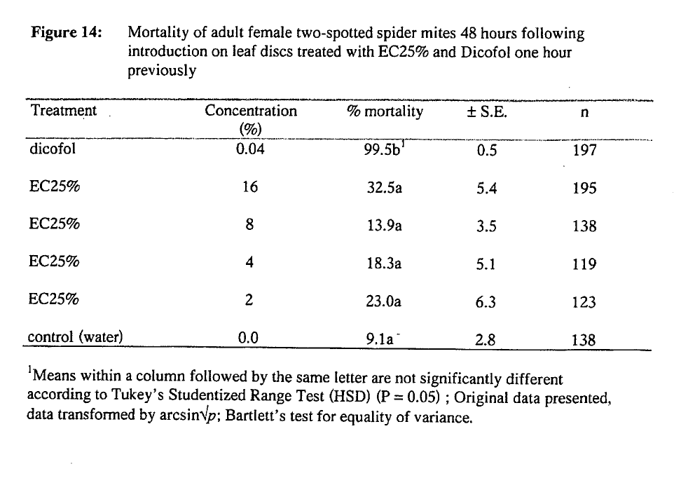

EC25% at 2, 4, 8 and 16% concentrations caused 23.0, 18.3, 13.9 and 32.5% mortality respectively to the adult spider mites when mites were introduced on bean leaves, 1 hr after treatment (FIG. 14). Dicofol's residual activity was significantly higher (99.5% mortality) than any of the EC25% concentrations.

EC25% was as effective as the insecticidal soap and synthetic acaricide abamectin to control adult twospotted spider mite and the European red mite. EC25% decreased egg hatch, but not as effectively as abamectin or neem oil. It may be important however to continue these investigations to determine the viability of emerged nymphs treated with the essential oil product because some botanicals, such as neem mixtures have shown growth-inhibiting properties to various pests (Rembald, 1989) and pulegone decreased larval growth of southern armyworm, Spodoptera eridania (Grunderson et al., 1985).

Furthermore we demonstrated that when adult mites are introduced one hour after treatment, the mortality rate was statistically comparable to that of the control (FIG. 14). A botanical such as EC25% may be an alternative to the more toxic or incompatible products. A contact acaricide with low residual activity can be used for treatments of localized infestations, before scheduled introductions of natural enemy populations or in absence of the natural enemy, i.e. treating at night in absence of diurnal parasitoids or predators.

Plant essential oils may be phytotoxic (Isman, 1999). The oil used for EC25% was evaluated on several edible and ornamental plants for its phytotoxic effects and results indicate that at the recommended dose, i.e. 0.5%, there were no observable effects on the leaves and flowers of tested plants (H. Chiasson, unpublished results).

### Example XI

### Insecticidal Efficacy of the Essential Oil Extract (Emulsifiable Concentrate Formulation)

Efficacy trials were conducted (laboratory and small-scale greenhouse trials) using the emulsifiable concentrate formulation of the present invention (EC at 25% of chenopodium oil comprising: 25% Essential oil extract; 2.5% Rhodopex CO-436; 2.5% Igepal CO-430; and 70% THFA) with the following organisms: the green peach aphid (Myzus persicae), the Western flower thrips (Frankliniella occidentalis), the greenhouse whitefly (Trialeurodes vaporariorium) as well as the parasitoid Encarsia formosa.

All bioassays were conducted in the laboratory of the Horticultural Research and Development Center (HRDC) of Agriculture and Agri-food Canada in Saint-Jean-sur-Richelieu, Quebec, Canada.

A. Contact bioassays in the laboratory and greenhouse using EC25% and commercially available bioinsecticides with the green peach aphid (Myzus persicae (Sulz.))

Laboratory Bioassay

Five concentrations (0.125, 0.25, 0.5, 1 and 2%) of formulation EC25% were compared to commercial preparations of Neem Rose Defense® at 0.5% (EC 90% hydrophobic Neem oil), Safer's Trounce® at 1% (EC 20% with 0.2% pyrethrin) and a water control. Each treatment was repeated 12 times and each replicate consisted of a 2 month old shoot (10-15 cm) of Verbena speciosa ‘Imagination’ placed in a plastic Aqua-Pick® (tube used by florist to keep stems of cut flowers wet) filled with 10 ml of water. Aqua Picks were secured on a block of Styrofoam placed on the bottom of a 1 l transparent plastic container modified with screened sides and top to permit aeration. Green peach aphids (Myzus persicae (Sulz.)) were collected in plastic containers from a rearing cage maintained in a greenhouse colony. Ten adults were transferred to each Verbena shoot. The shoot was sprayed at 8 psi under an exhaust chamber for about 15 seconds (long enough to cover the whole shoot) with a VEGA 2000® paintbrush sprayer equipped with a 20 ml reservoir (Thayer & Chandler Co., Lake Bluff, Ill., USA). Each shoot and plastic container was then stored in a growth chamber at 24° C., 65% R.H. and 16 L:8 N photoperiod. The entire procedure was repeated four times.

Mortality was evaluated 48 hours following treatment by probing the aphid for movement with a small brush; absence of movement was recorded as dead. To evaluate the relative efficacy of EC25%, Neem Rose Defense® and Safer's Trounce®, percentage mortality data were transformed to arcsin√p and subjected to ANOVA analysis using SAS® software (SAS Institute 1988). LC₅₀ and LC₉₀ were calculated using mortality results by PROBIT analysis using POLO-PC® software (LaOra Software 1994). Product concentrations (%) were used because data on quantity of active material deposited were not available.

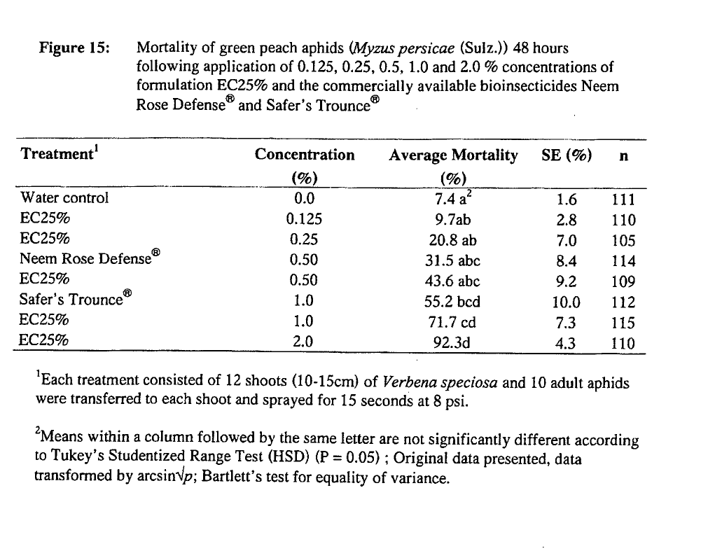

Results show that EC25% at 2.0% concentration was more effective (92.3% mortality) at controlling the green peach aphid than EC25% at 1% concentration (71.7%) and Safer's Trounce® (55.2%) though not significantly (FIG. 15 ). This lack of distinction between treatments may be due to the low number (n) of aphids tested. Treatments with EC25% at concentrations of 0.5% and less and with Neem Rose Defense® resulted in <50% mortality of the aphids and results were not significantly different to those obtained with the water control.

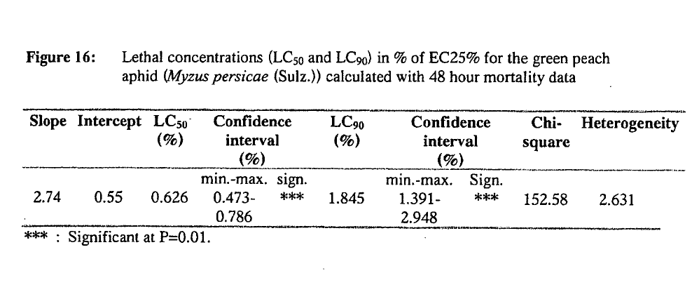

The LC₅₀ and LC₉₀ of EC25% for the green peach aphid was 0.63 (in % concentration) (Confidence Interval 0.47%-0.79%) and 1.84% (Confidence Interval of 1.39%-2.95%) respectively (FIG. 16).

Greenhouse Bioassay

Three concentrations (0.25, 0.5 and 1%) of formulation EC25%, Neem Rose Defense® at 0.5% (EC 90% hydrophobic Neem oil), Safer's Trounce® at 1% (EC 20% with 0.2% pyrethrin) and a water control were tested with the green peach aphid (Myzus persicae (Sulz.)). Fifteen plants (replicates) of two month old Verbena speciosa ‘Imagination’ (10-15 cm) grown in small plastic insertions cells (used for potting plants) filled with Pro-Mix BX® were used for each treatment. Each insertion cell was glued to the bottom of a 1 l transparent plastic container with screened sides and top, to permit aeration. Green peach aphids were collected in plastic containers from a rearing cage maintained in a HRDC greenhouse and ten adults were transferred to each plant. The whole plant was sprayed for 15 seconds on average, at 8 psi under an exhaust chamber with a VEGA 2000® paintbrush sprayer equipped with a 20 ml reservoir (Thayer & Chandler Co., Lake Bluff, Ill., USA). Spraying was done three times over the course of the experiment, i.e. on days 0, 7 and 14. Containers with the sprayed plants were kept in a greenhouse under shade for the duration of the experiment.

Counts were done on days 7, 14 (prior to spraying) and on day 21 by dismantling five of the fifteen replicates in each treatment. Aphids were individually counted when numbers were small (<50). For larger numbers, plants were shaken over a clear 250 ml container filled with soapy water over a black and white grid to evaluate the number of aphids present. Plant leaf surface (cm²) was measured with an area meter LI-3100® (LI-COR Inc., Lincoln, Nebr., USA) and counts were averaged to number of aphids/cm² for each treatment and transformed to square root (x+0.5) for ANOVA analysis with SAS® software to evaluate the efficacy of the different treatments. Counts within treatments did not differ significantly (P=0.3647) from one sampling day to the other, so results within treatments were pooled and averaged for the whole experiment.

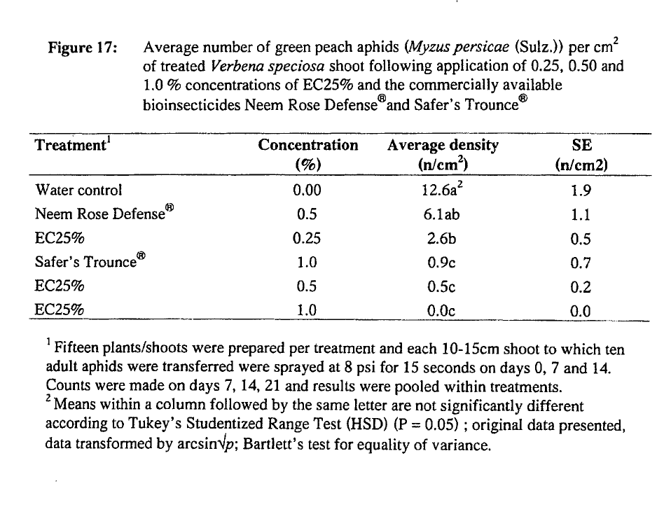

All concentrations of EC25% and Safer's Trounce® were more effective in controlling the aphids than the water control (FIG. 17 ). EC25% at 0.5% and 1.0% and Safer's Trounce were significantly more effective in reducing the number of aphids/cm² than Neem Rose Defense® and EC25% at 0.25%. Both 0.5% and 1.0% EC25% concentrations were more effective (0.5 aphids/cm² and 0.0 aphids/cm² respectively) than Safer's Trounce® (0.9 aphids/cm²) though not significantly.

B. Contact bioassays in the laboratory and greenhouse with the western flower thrips (Frankliniella occidentalis (Perg.)) using EC25% formulation and two commercially available bioinsecticides.

Laboratory bioassay

Six concentrations (0.05, 0.18, 0.125, 0.25, 0.5 and 1%) of formulation EC25%, Neem Rose Defensee at 0.7% (EC 90% hydrophobic Neem oil), Safer's Trounce® at 1% (EC 20% with 0.2% pyrethrin) and a water control were tested with the Western flower thrips (WFT: Frankliniella occidentalis (Perg.)). WFT were collected in plastic containers by tapping infested Lima bean leaves over white paper. Ten WFT (either adults or 3^{rd} or 4^{th} instar nymphs) were transferred to a closed 250 ml transparent plastic container. Wet dental cotton was inserted through the lid for use as a water source. Four replicates were prepared for each treatment. Containers were sprayed at 6 psi under an exhaust chamber for 15 seconds with a VEGA 2000® paintbrush sprayer equipped with a 20 ml reservoir (Thayer & Chandler Co., Lake Bluff, Ill., USA). Containers were weighed just before and after spraying to calculate the amount of active ingredient deposited in mg/cm². Containers were then stored in a growth chamber at 24° C., 65% R.H. and 16 L:8 D photoperiod. The entire procedure was repeated four times.

Mortality was evaluated 24 hours following treatment under a binocular scope by probing WFT with a small brush. Absence of movement was recorded as dead. The efficacy of EC25% was compared to Neem Rose Defense® and Safer's Trounce® and data were transformed by arcsin√p and subjected to ANOVA analysis using SAS® software (SAS Institute 1988). The LC₅₀ and LC₉₀ (in mg/cm² of active ingredients) were calculated mortality results by PROBIT analysis using POLO-PC® software (LaOra Software 1987).

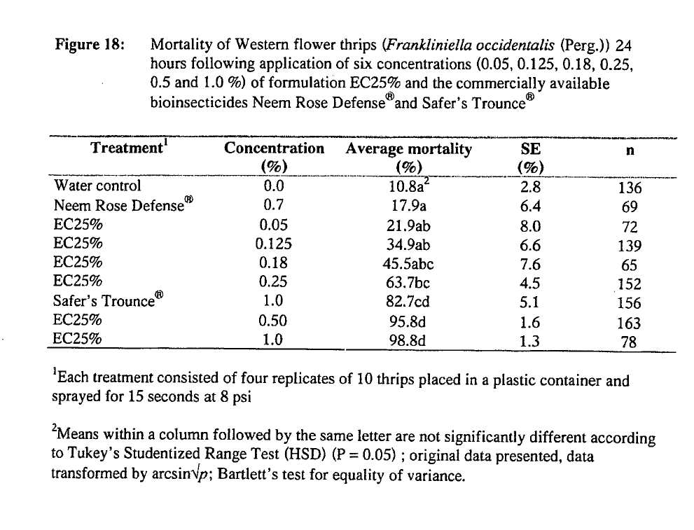

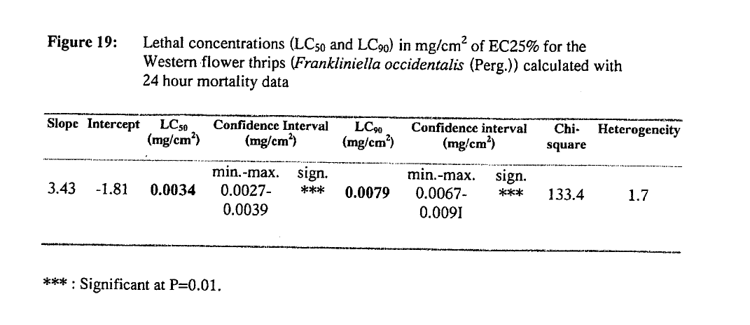

Formulation EC25% at 0.5% and 1.0% were significantly more effective (98.8% and 95.8% mortality respectively) in controlling the WFT than all other treatments except for Safer's Trounce® (82.7% mortality) (FIG. 18). EC25% at 0.25% caused significantly more mortality (63.7%) than the control (10.8%) but all remaining treatments did not. The LC₅₀ and LC₉₀ of EC25% for thrips was determined as 0.0034 mg/cm² (Confidence Interval: 0.0027-0.0039 mg/cm²) and 0.0079 mg/cm² (Confidence Interval: 0.0067-0.0099 mg/cm²) respectively (FIG. 19).

Greenhouse Bioassay

Two concentrations (0.25% and 1%) of formulation EC25%, Neem Rose Defense® at 0.7% (EC 90% hydrophobic Neem oil), Safer's Trounce® at 1% (EC 20% with 0.2% pyrethrin) and a water control were used to evaluate their relative efficacy in controlling the Western Flower thrips (WFT: Frankliniella occidentalis (Perg.)) in a greenhouse setting. Ten 10 day-old Lima bean plants (Phaseolus sp.) were prepared for each treatment. One leaf and the cotyledons of each plant were removed to keep only one leaf per plant grown in Pro-Mix BX® in a plastic insertion cell (used for potting plants) glued to the bottom of a clear plastic container (1 l) with screened sides and top. WFT were collected in small plastic containers by tapping infested bean leaves over white paper and lifted with a small brush. Ten adult thrips (or 3^{rd} or 4^{th} instar larvae) were transferred on each single leaf of each plant/insertion cell which were sprayed to drip point at 6 psi under an exhaust chamber with a VEGA 2000® paintbrush sprayer equipped with a 20 ml reservoir (Thayer & Chandler Co., Lake Bluff, Ill., USA). Spraying was done on days 0, 8 and 14. Each replicate/plastic container was then kept in a greenhouse under shade for the duration of the experiment.

Counts were made on days 8 and 14 (prior to spraying) and on days 21 and 28. All live stages present on the whole plant were counted under a binocular scope and the leaf surface was measured by comparing it to a series of pre-measured hand-made leaf-size patterns. On the last day of the experiment (day 28), the leaf was cut and its surface was measured with an area meter LI-3100® (LI-COR Inc., Lincoln, Nebr., USA). Counts were calculated as average number of thrips/cm² per treatment. In order to compare treatments, average counts were then calculated as a percentage of thrips present on the control plants:
(n/cm² on treated plants/n/cm² on control plants)×100

The control treatment therefore had a value of zero and other treatments had positive or negative values indicating that more or less thrips were present respectively in relation to the control treatment.

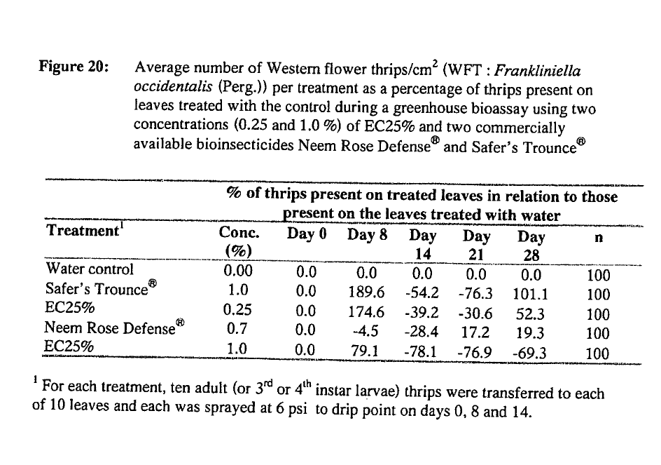

At the end of the experiment on day 28, leaves treated with EC25% at a concentration of 1.0% had 69.3% less WFT than leaves treated with the control while leaves treated with Safer's Trounce® had 101.1% more WFT (FIG. 20). Leaves treated with Neem Rose Defense® had slightly more thrips (19.3%) than the control on day 28. Leaves treated with EC25% at 0.25% concentration had 52.3% more thrips than the control on day 28.

C. Contact bioassay in the laboratory with the greenhouse whitefly (Trialeurodes vaporariorium (Westw.)) using EC25% and commercially available insecticides

Laboratory Bioassay

Five concentrations (0.0625, 0.125, 0.25, 0.5 and 1%) of formulation EC25%, Neem Rose Defense® at 0.7% (EC 90% hydrophobic Neem oil), Safer's Trounce® at 1.0% (EC 20% with 0.2% pyrethrin), Thiodan® at 0.044% (50 WP) and a water control were used to evaluate their relative efficacy in controlling the greenhouse whitefly (Trialeurodes vaporariorium (Westw.)). Whitefly adults were collected with an insect aspirator from HRDC greenhouses and glued to a black 5 cm×7,5 cm plastic card sprayed with Tangle-Trap® (Gempler's Co.) by emptying the aspirator over the card to obtain at least 20 adults per card. Cards were observed before spraying under the binocular scope to remove all dead and immobile whiteflies. Only active whiteflies were kept for the experiment. Four cards were used per treatment. Each card was sprayed at 6 psi with 300 μl of emulsion using a BADGER 100-F® (Omer DeSerres Co., Montreal, Canada) paintbrush sprayer mounted on a frame at a distance of 14.5 cm from the spray nozzle in an exhaust chamber. Cards were weighed immediately before and after spraying to calculate the amount of active ingredient deposited in mg/cm². Cards were allowed to dry under the exhaust chamber and then placed sideways on a Styrofoam rack in a closed clear plastic container of 5 L with moistened foam on the bottom to keep humidity high (>90% R.H.). The plastic container was stored in a growth chamber at 24° C. and 16 L:8 D photoperiod. This procedure was repeated three times. Mortality was evaluated 20 hours following treatment by gently probing the whitefly with a single-hair brush under the binocular microscope. Absence of movement (antennae, leg, wing) following probing was recorded as dead. Relative efficacy of EC25% and the two commercially available bioinsecticides, Neem Rose Defense® and Safer's Trounce®, and the synthetic insecticide Thiodan®, were compared by transforming mortality data to arcsin√p and then subjecting to an ANOVA analysis using SAS® software (SAS Institute 1988). LC₅₀ and LC₉₀ (in mg/cm² of active ingredients) were calculated by PROBIT analysis using POLO-PC® software (LaOra Software 1987).

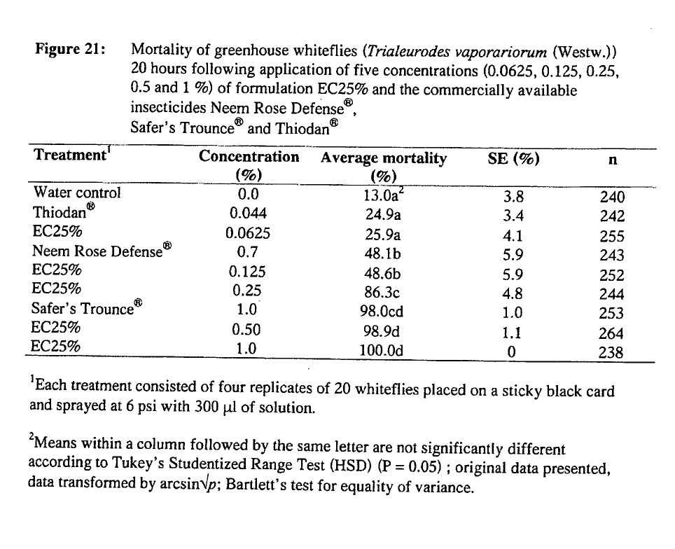

Formulation EC25% at concentrations 0.5% and 1.0% were significantly more effective (98.9% and 100.0% mortality respectively) at controlling the greenhouse whitefly than all other treatments except for Safer's Trounce® (98.0% mortality) (FIG. 21). Formulation EC25% at 0.125% concentration and Neem Rose Defense® were significantly more effective than the control treatment but significantly less effective than EC25% at 0.25, 0.5 and 1.0% concentrations and Safer's Trounce®. Thiodan and EC25% at 0.0625% concentration were as effective as the control treatment.

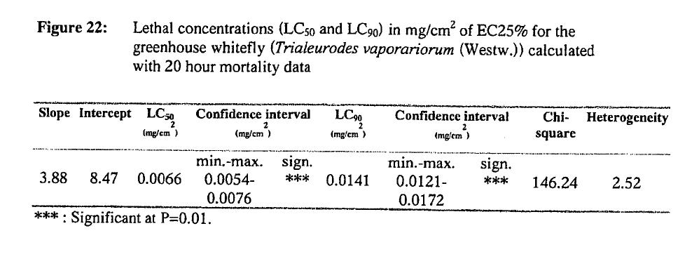

LC₅₀ and LC₉₀ were 0.0066 mg/cm² (conf. int: 0.0054-0.0076 mg/cm²) and 0.0141 mg/cm² (conf. int: 0.0121-0.0172mg/cm²) respectively (FIG. 22).

D. Contact bioassay in the laboratory with the parasitoïd (Encarsia formosa) using EC25% and commercially available bioinsecticides

Laboratory Bioassay

Four concentrations (0.0625, 0.125, 0.25 and 0.5%) of formulation EC25%, Neem Rose Defense® at 0.7% (EC-90%), Safer's Trounce® at 1.0% (EC 20.2%) and a water control were tested with the parasitoïd Encarsia formosa (EF) (obtained from Koppert Co. Ltd). EF were kept in a growth chamber at 24° C., 16 L:8 N photoperiod and 65% R.H. until emergence. Sixty newly emerged adult EF were transferred with a mouth aspirator into plastic Solo® cups of 20 ml. Cups were sprayed at 6 psi under an exhaust chamber with 250 ml of solution with a Badger 100-F® paintbrush sprayer (Omer de Serre Co., Montreal, Canada) mounted on a frame at a fixed distance of 14.5 cm. Solo® cups were weighed just before and after spraying to calculate the amount of active ingredient deposited in mg/cm². Once sprayed, the EF were gently transferred with a small brush from the Solo cups to small clear plastic Petri dishes (10 EF/Petri) lined with a filter paper wetted with a 5% sugar solution as a food source. Four replicates were prepared for each treatment. The Petri dishes were then placed in a tray and stored in a growth chamber at 24° C., 65% R.H. and 16 L:8 D photoperiod. The entire procedure was repeated three times.

Mortality was evaluated 24 hours following treatment under a binocular scope by observing the EF. Absence of movement was recorded as dead. The effect of EC25% was compared to Neem Rose Defense® and Safer's Trounce® using mortality data transformed by arcsin√p and subjected to ANOVA analysis using SAS® software (SAS Institute 1988).

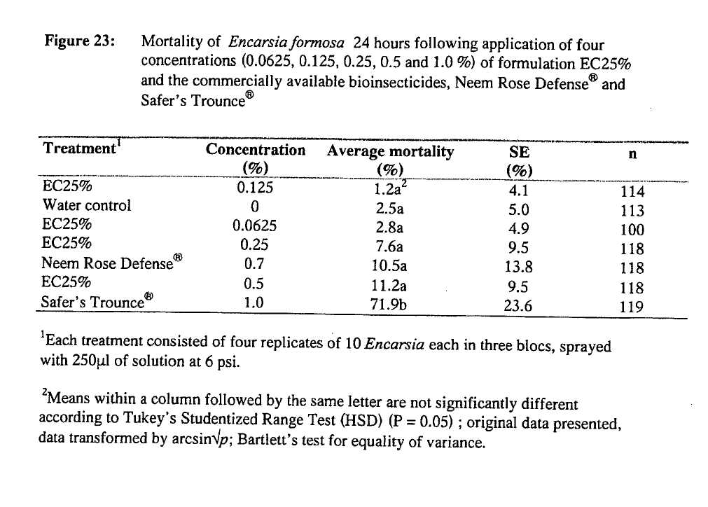

All EC25% formulations at concentrations ranging from 0.0625 to 0.5% were significantly less effective than Safer's Trounce® at 1% (71.9%) (FIG. 23). Results from all concentrations of EC25% and Neem Rose Defense® formulations were not significantly different than the control. These results indicate that the recommended dose (0.5%) of EC25% can be safely used with the biological control agent, Encarsia formosa.

### Example XII

### Fungicidal Efficacy of the Essential Oil Extract (Emulsifiable Concentrate Formulation)

A. Effect of EC on Gray Mold Caused by the Plant Pathogen, Botrytis cinerea on Greenhouse Tomatoes

Tomatoes (cv Trust) were sown and grown according to practices generally used for the production of greenhouse tomatoes. About two months after sowing, lesions are made on the stem (5 lesions/plant) and inocuated with a suspension of 3×10⁶ spores of B. cinerea, 1 ml per lesions. The following treatments were evaluated: 1) control (−) with water only; 2) control (+) with B. cinerea; 3) Nova® (0.3 g/l); 4) Iprodione® (Rovral 1.0 g/l), 5) EC25% (comprising: 25% Essential oil extract; 2.5% Rhodopex CO-436; 2.5% Igepal CO-430; and 70% THFA) at a concentration of 0.125%; 6) EC25% at a concentration of 0.50%; 7) EC25% at a concentration of 0.75%; 8) M. ochracea (P130A) (5×10⁸ spores/ml); 9) Rootshield® (0.6 g/l). The experimental design was a randomized block design with 8 blocks. Treatments were applied 1 and 8 days following inoculation of lesions with B. cinerea.

Disease symptoms were evaluated according to the following severity index: 1=absence of symptoms, 2=necrosis (browning of tissue) around the lesion, 3=advanced necrosis and presence of spores on necrotic zones, 4=advanced necrosis, presence of spores on necrotic zones and the start of canker formation, 5=well developed canker and death of plant. Severity of symptoms was estimated 3 and 5 weeks after plant inoculation. The experiment was repeated and the effect of treatment was analyzed with an analysis of variance (ANOVA) and means were compared with a Least Significant Difference test (LSD).

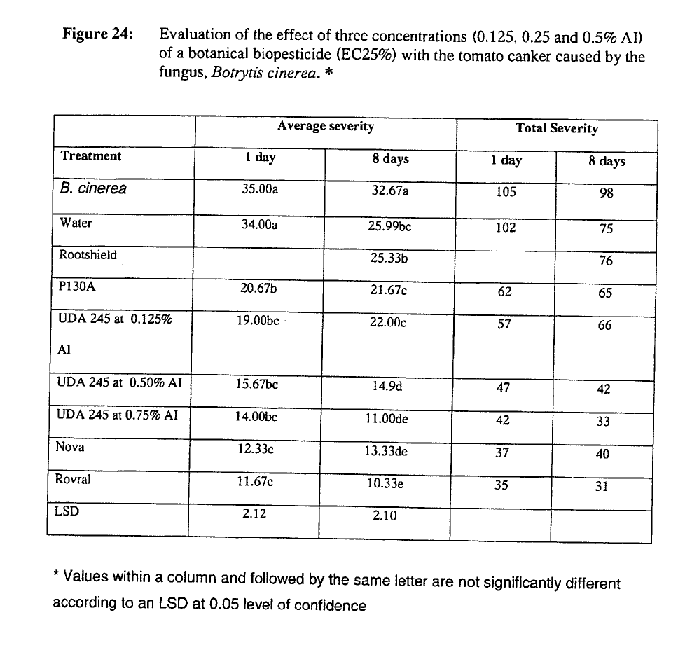

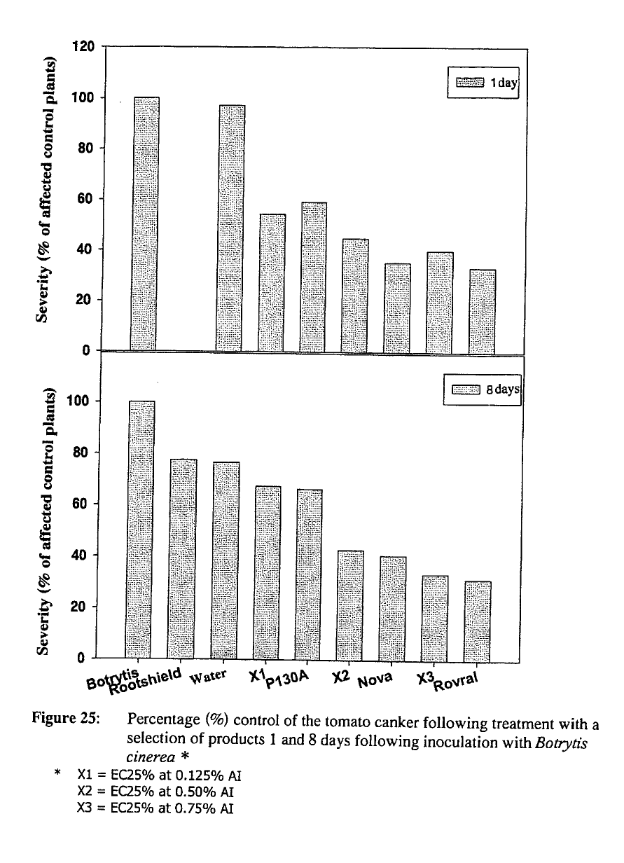

Statistical analyses demonstrated that there was a significant difference in efficacy between treatments (FIG. 24). Control plants demonstrated average severity of disease symptoms on 26 of the 35 control plants. Plants from all other treatments presented a significantly lower level of severity than the control plants one day after inoculation or the first day of treatment. The two microbial fungicides Rootshield and P1304 did not significantly control the disease with an average severity between 25.33 and 21.67 8 days after treatment (FIG. 24). Plants treated with the botanical biopesticide EC25% was less effective in controlling the disease symptoms than the commercial products, Nova and Rovral. However, all three concentrations of EC25% (0.125, 0.25 and 0.5% AI) decreased disease symptoms with severity indexes being 60, 44 and 37% lower respectively on plants treated with EC25% than the control plants. Severity indexes were reduced by 38 and 32% with Nova and Rovral respectively (FIG. 25).

B. Effect of EC25% on Powdery Mildew Caused by the Plant Pathogens, Erysiphe cichoracearum and Sphaerotheca fuliginea on Greenhouse Cucumbers

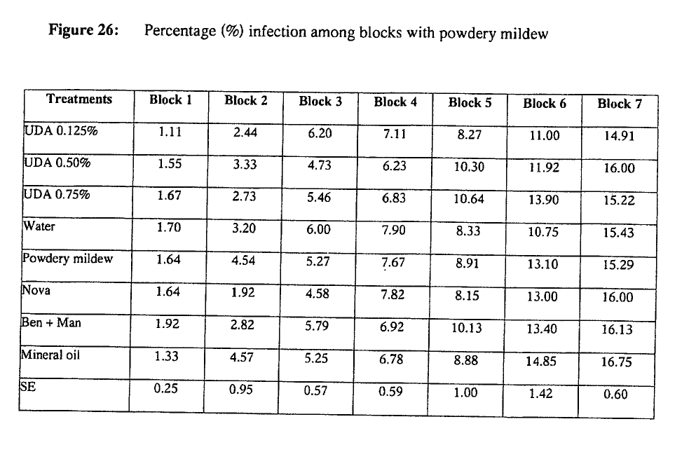

The fungus responsible for powdery mildew are obligatory parasites incapable of survival in the absence of the host. In order to produce inoculum necessary for inoculation, infested cucumber leaves are collected from a grower in July of 2002. Conidia present on these leaves were transferred on leaves of cucumber plants grown in an experimental greenhouse, one of to two months after sowing the cucumber plants (cv Straight 8). The following treatments were evaluated: 1) negative control treated with water; 2) positive control treated with the disease organisms, E. cichoracearum and S. fliginea; 3) Nova® (0.3 g/l); 4) Benlate®+Manzate® (1.2 g/l and 4. g/l); 5) EC25% (comprising: 25% Essential oil extract; 2.5% Rhodopex CO-436; 2.5% Igepal CO-430; and 70% THFA) at 0.125% AI; 6) EC25% at 0.50% AI; 7) EC25% at 0.750% AI; 8) Mineral oil. The experimental design was a complete randomized block design with 7 blocks. Since it is almost impossile to inoculate an obligatory fungal parasite in an known or measurable quantity of inoculum, there is always variability in the level of infection of the plants. A first evaluation of powdery mildew was made on all plants inoculated in the greenhouse. The percentage of infection was analyzed in order to group 8 plants presenting the same level of infection (SE <1.5, see FIG. 26).

Severity of powdery mildew was also evaluated on each individual leaf with the use of a severity index 7 days after treatment applications and reported as average severity for each plant. The entire experiment was repeated a few days later. The effect of treatment was analyzed with an analysis of variance (ANOVA) and means were compared with a least significant difference test (LSD).

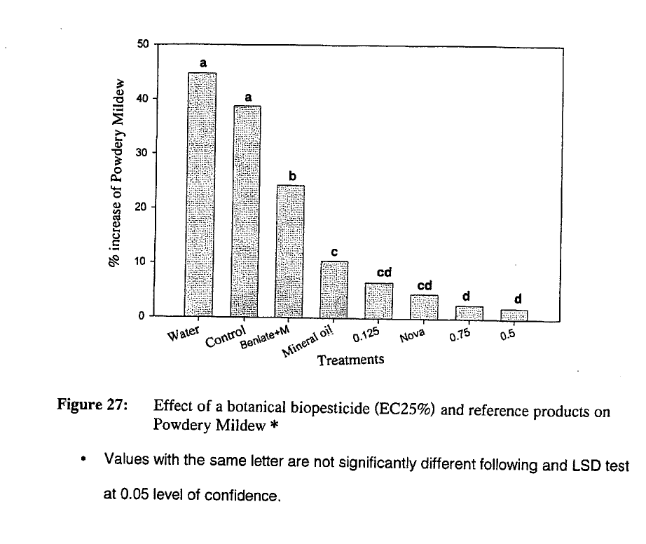

Statistical analysis demonstrate that there is a difference of effect between treatments. (FIG. 27). In fact, an increase of 45 and 39% of disease incidence was observed on control plants (water and disease). All plants treated with the various products presented a significantly lower increase in percentage of disease severity compared to the control plants. The botanical biopesticide EC25% was as effective as the commercial fungicide Nova in controlling the disease. At concentrations of 0.50 and 0.75%, EC25% demonstrated better control than Nova, permitting an increase of disease symptoms of only 2 and 2.5% respectively for both concentrations compared to 4.5% for Nova. However phytotoxicity was observed on plants treated with EC25% at those two concentrations

### Example XIII

### Effect of Essential Oil Extract for Controlling Soil Inhabiting Pests

A. Effect of Soil Treatments with EC25% at 0.5% on Soil Emergence of Adult Western Flower Thrips

Lettuce plants were transplanted in plastic pots of 12 cm of diameter and grown for six weeks in a greenhouse. The plants were heavily infested with all stages of thrips Frankliniella occidentalis Pergande and were watered and fertilized as required by good management practices. Then, the lettuce plants were cut at the soil level and the pots were sprayed with a preparation of EC25% (comprising: 25% Essential oil extract; 2.5% Rhodopex CO-436; 2.5% Igepal CO-430; and 70% THFA) at 0.5 concentration of active ingredients. Five volumes of EC25% were applied, 25 ml, 16.7 ml, 13.3 ml, 10 ml, and 6.67 ml per pot representing volumes of 1500 l/ha, 1000 l/ha, 800 l/ha, 600 l/ha and 400 l/ha respectively. A total of 10 pots were sprayed per volume and control pots were sprayed with 25 ml of tap water. The application was made using a badger sprayer connected to an air outlet to provide the required air pressure for pesticide application. Immediately after treatments, the pots were covered with a plastic petri dish cover of 14.5 cm diameter. The inner surface of the cover was smeared with a thin layer of tanglefoot insect glue and the joints were completely sealed with parafilm to prevent the escape of emerging adults from the soil pot or the introduction of foreign into the dish. Treatments were applied on 7 July 2002 and counts of emerging adults thrips were made 3, 5, 7, 10 and 12 days after treatments. At each observation time, emerging adults were removed from the glue and the cover was replaced on the top of the plastic pot and sealed with parafilm.

Counts of emerging adults over a period of 2 weeks after treatments were analysed using an ANOVA and means for each treatment were separated using a Fischer LSD test.

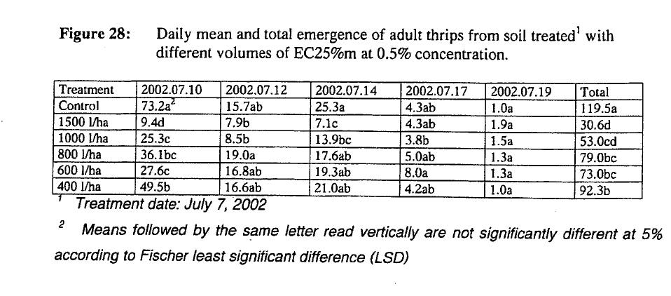

Most of the adult thrips emerged during the first week following treatment and this corresponds with the duration of pupal life that varies between 2 and 8 days according to the ambient temperature (FIG. 28). Effect of treatment on the total number of adult thrips emergence from the soil was statistically highly significant ((F=5.71; dl=5; P<0.0001).). All treatments significantly reduced thrips emergence compared to the control. At 3 days after treatment, an important decrease in the number of emerging adult thrips was observed in the 1500 l/ha treatment compared with all other treatments. At all other dates, the 1500 l/ha treatment was the best treatment and caused the greatest reduction in adult thrips emergence.

B. Using EC25% and Microemulsion Formulations for Controlling Soil-Inhabiting Pests of Turf

i) Laboratory Tests using EC25% with the European Chafer, Rhizotrogus maialis

The formulation EC25% (comprising: 25% Essential oil extract; 2.5% Rhodopex CO-436; 2.5% Igepal CO-430; and 70% THFA) was tested with third instar R. majalis. The insects were collected from Arnprior ON, and were kept in containers with soil at 4° C. until trials were initiated. Grass was grown in plastic seedling trays divided into 6 compartments. Soil within each compartment consisted of 1 part potting soil: 1 part sand to a depth of approximately 5 cm. The grass seedling mix consisted of Kentucky Blue 65%, Annual Rye Grass 20% and Fescue 15% and was distributed at 1.5 g per compartment 2-3 weeks prior to the addition of the insects. Ten third instar R. majalis were placed into each compartment so for a total of 60 larvae per tray. Three replicate trays were used for two separate experiments. One to two days after the larvae were added to the trays, treatment was initiated. Each of the EC25% treatment concentrations; 0.0625, 0.125, 0.25, 0.5 and 1%, were prepared in 100 mL of water by the addition of 0.25, 0.5, 1, 2 and 4 mL of stock EC respectively. The control was an EC blank, 0.375 mL of formulation excluding essential oil. The trays were watered once per day for the seven day period after treatment. After one week each compartment was sampled to determine the survival of the larvae. Samples of the soil were taken from each compartment after both trials and analysed for both pH (ASTM, 1989) and organic matter content (ASTM, 1987).

Probit analysis was used with the data from the two European chafer trials in order to determine the LC₅₀ values for EC25%. Comparison of the LC₅₀ values within and between each trial was conducted using a z-test.

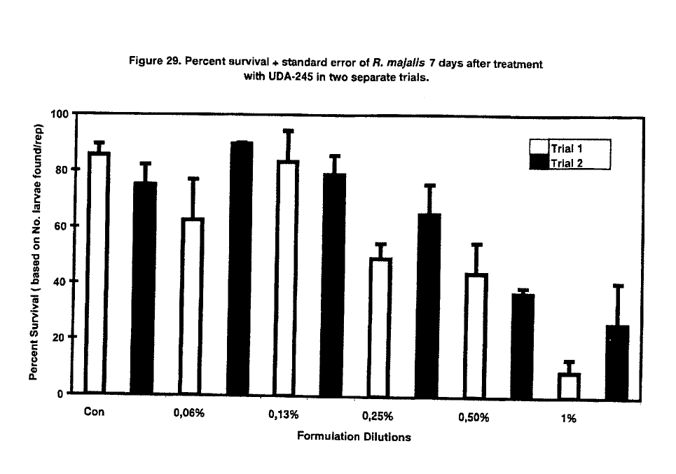

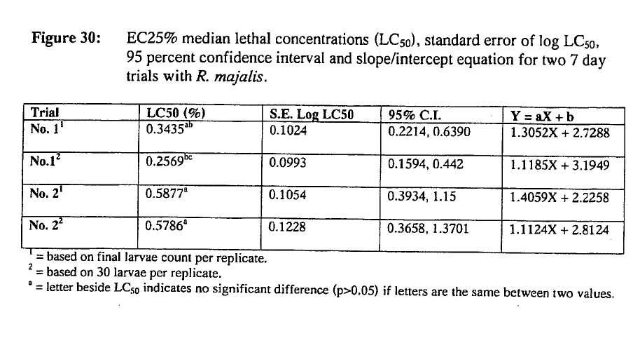

Ninety percent (90%) R. majalis larvae survived at the 0.0625% concentrations of EC25% and 10% survived at 1% EC25% (FIG. 29). Percent mortality from both trials were used to calculate LC₅₀ values (FIG. 30). Two different values were calculated for each trial since not all of the larvae were accounted for at the end of the exposure period.

The first LC₅₀ for each trial is based on the mortality within the actual number of larvae found in each treatment replicate at the end of the test (FIG. 30). The second LC₅₀ is calculated based on the assumption that there were 10 larvae present at the beginning and end of the experiment within each treatment replicate but due to possible deterioration of the organism it was not identified or located.

A comparison of the LC₅₀ using a z-test indicated that within each trial there was no significant difference between the calculated LC₅₀s (z<0.885, z critical=1.96) Between trials it was determined that there was no significant difference (z=1.587, z critical=1.96) when comparing the first LC₅₀ (based on the actual number of larvae found). When the LC₅₀s based upon 30 larvae per treatment were compared between trials a significant difference (z=2.233, critical z=1.96) was detected. A mean LC₅₀ and standard error for the two trials was calculated as 0.466±0.122% EC25% by averaging the two LC₅₀s from trial one and two and these were not significantly different.

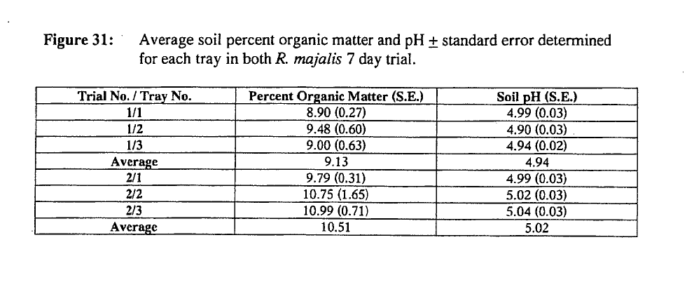

Soil Organic Matter Content and pH. The soil organic matter content was determined to range from 8.9 to 11 with an average of 9.1 in the first trial and 10.5 in the second (FIG. 31). The pH of the soil ranged from 4.9 to 5.0 with a mean of 4.94 in the first and 5.02 in the second trial. These values were consistent with those measured in previous R. majalis trials.

ii) Field Tests using Microemulsion Formulations with the European Chafer, Rhizotrogus majalis

The objective of this study was to measure the effect of broadcast applications of two microemulsion formulations (MEF), MEF1 and MEF2 of an essential oil extract from Chenopodium ambrosioides, on the presence of the European chafer, Rhizotrogus majalis.

The microemulsion formulations tested comprised the following:

| Ingredient | MEF 1 | MEF 2 |
|---|---|---|
| Chenopodium extract | 50 | 50 |
| Rhodafac RE-610 | 10 | 10 |
| Ammonyx LO | 4 | 4 |
| Stepanol WAC | 1 | — |
| Water | 35 | 35 |

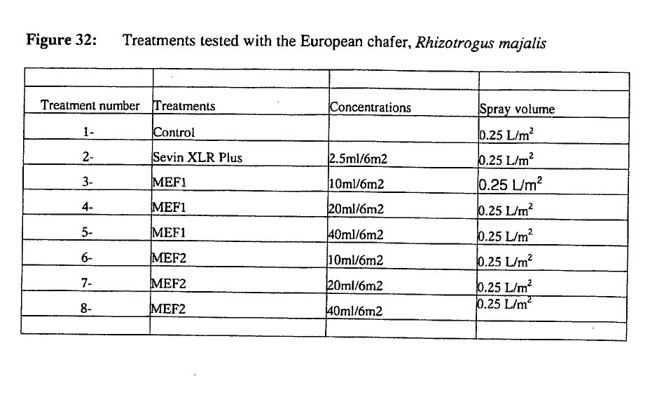

The experiment was conducted on lawn at the Guelph Turfgrass Institute (GTI), Ontario on turfgrass that was mowed once a week and received irrigation. Experimental design was a randomized complete block design with 4 replications and 8 treatments. Plots were 3 m×3 m and treatments tested are listed in FIG. 32. Treatments were applied on Aug. 27, 2002, immediately after pre-treatment assessment of the European chafer, with a sprayer in water at 0.25 L/m². The European chafers were at the second instar when treatments were applied and at the third instar by the end of the experiment. The live European chafers were counted using a golf course hole-cutter (15.2 cm, ‘turf mender’). Five plugs were taken from each plot. Once removed, plugs were broken up and the number of chafers was recorded. The last chafer count was done using a sod-cutter: three turf strips (0.3 m×3m) across each plot. The turfgrass sod was removed and chafers near the soil surface were counted. Counts were made at 5, 15 and 25 days after treatment on 2, 15, and 23 Sep. 2002, respectively.

Analysis of variance (ANOVA) on the number of total European chafes alive was parformed by the General Linear Model Procedure of SAS statistical software (SAS Institute 1990). Fisher's Least Significant Difference test (LSD) was used for comparison of treatment means when the F-test was significant (P=0.05).

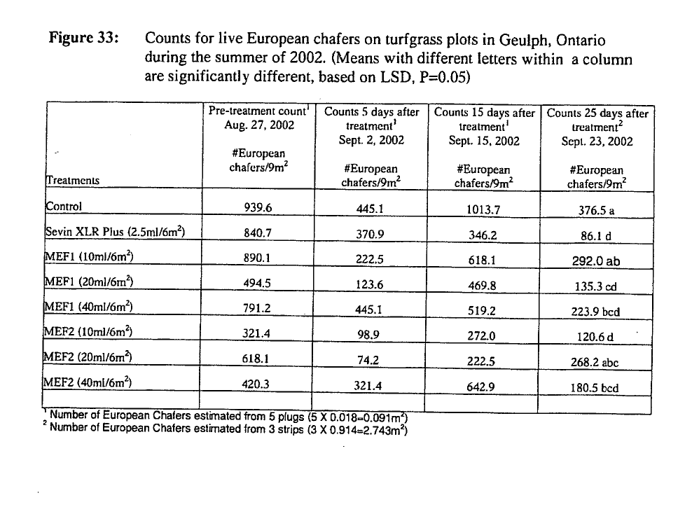

Number of European chafers was not significantly different between plots prior to treatment application. On September 23 (25 days after the application), all insecticide treatments significantly reduced the number of chafers compared to the untreated control except treatments MEF1 (10.0 ml/m²) and MEF2 (20.0 ml/m²) (F=4.25; P<0.00046) (FIG. 33). European chafer counts for both treatments were significantly higher than counts on plots treated with SEVIN XLR PLUS.

No significant difference in the number of chafers in turfgrass plots was observed on September 2 (5 days after application) and on September 15 (15 days after application). For both dates, all treatments reduced chafers compared to the untreated control lower, the statistical model was not significant mainly because of the fact that European chafers were counted on a smaller area at these two dates.

iii) Field Tests with the Microemulsion Formulations MEF1 and MEF2 and the Hairy Chinch Bug, Blissus leucopterus

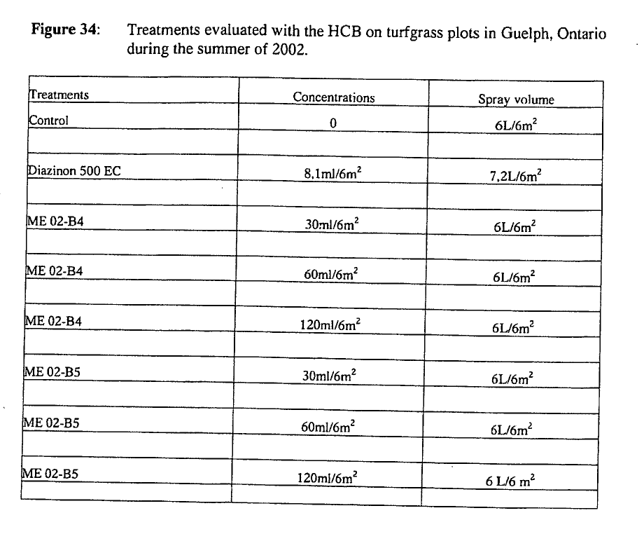

The experiment was conducted on a commercial lawn in Guelph, Ontario on low maintenance turfgrass that was mowed once a week and received no irrigation. Experimental design was a randomized complete block design with 4 replications and 8 treatments. Plots were 2 m×3 m and treatments tested are listed in FIG. 34. Treatments were applied on Jul. 30, 2003, immediately after pre-treatment assessment of the HCB, with a sprayer in water at 1 L/m². Most of the HCB population was at the third instar. HCB were counted using the flotation method. A cylinder (346 cm²) was inserted into the turf to a depth of about 3 or 4 inches and was flooded to the brim with water. After 5 minutes, live HCB floating to the surface were counted. Three counts per plot were made (by the same person) and the total HCB per plot was used for statistical analysis. HCB were counted 5, 15 and 25 days after treatment on August 4, 12 and 24, respectively.

Analysis of variance (ANOVA) on the number of total live HCB was performed using the General Linear Model Procedure of SAS statistical software (SAS Institute, 1990). Fisher's Least Significant Difference test (LSD) was used for comparison of treatment means when the F-test was significant (P=0.05).

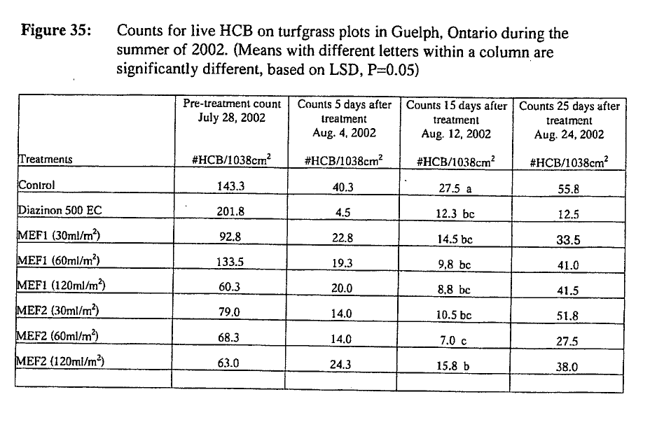

Number of HCB was not significantly different between plots prior to treatment application. All insecticide treatments significantly reduced the number of HCB compared to the untreated control on August 12, 15 days after the application (F=6.13; P<0.0005) (FIG. 35). There were no differences between results with the Diazinon treatment and results with the two formulations. At this date, treatment MEF2 (20.0 ml/m²) had significantly more insects than treatment MEF2 (10.0 ml/m²).

No significant difference in the number of HCB in turfgrass plots was observed on August 4 (5 days after application) and on August 24 (25 days after application). For both dates, all treatments reduced HCB compared to the untreated control.

### Example XIV

### Effect of Essential Oil Extract on Beneficial Pests

A. Direct Toxicity of the Essential Oil Extract on Predatory Mites Amblyseius fallacis and Phytoseiulus persimilis

The purpose of this study was to evaluate the direct toxicity of the EC25%, a botanical biopesticide with two predaceous mites Amblyseius fallacis, a natural regulator of mites in integrated control orchards and Phytoseiulus persimilis, a known mite predator for the control of the twospotted mite in vegetable crops grown under glasshouses in Quebec and elsewhere. The suitability of EC25% as a primary tool in IPM of greenhouse crops would therefore be determined.

Rearing of Tetranychus urticae and Amblyseius fallacis

The phytophagous mite, Tetranychus urticae has been reared on common bean plants (Phaseolus vulgare) for several years at the Horticultural Research and Development Center, St. Jean-sur-Richelieu, Quebec. The beans were sown at high densities of 40 to 50 plants per tray (39 cm×30 cm). Colonies of T. urticae were kept in a growth chamber set at 25° C., 75% HR and 16 L photoperiod.

The predaceous mite Amblyseius fallacis was maintained on Tetranychus urticae and kept in a greenhouse set at 25° C., 75 HR and 16 L photoperiod. A fan placed in front of the cage containing both Amblyseius fallacis and the twospotted spider mite provided continuous air flow to the colonies. Trays containing bean plants infested with the twospotted spider mites were added regularly to provide sufficient food to the predator colonies.

Rearing of Phytoseiulus persimilis

Colonies of Phytoseiulus persimilis were bought from Koppert Canada and reared in the laboratory in the same conditions as for A. fallacis. The colonies originating from the shipment were maintained and acclimatized in a growth chamber set at 25 C, 70-85% RH and 16:8 (light/darkness) for two weeks.

Contact Toxicity Assay

The bioassays were carried out in Petri dishes using a leaf disc method. A wet sponge was placed in a plastic Petri dish (14 cm diameter and 1.5 high) and rings of apple leaf (cv. McIntosh; 3.5 cm of diameter) were cut and placed upside down on the surface of a water-saturated sponge. Sufficient numbers of all stages of the twospotted spider mite Tetranychus urticae Koch were then brushed onto each leaf disc. A total of five leaf discs were put in a Petri dish and each Petri dish represented one replicate. Ten replicates per treatment were prepared over a period of three weeks.

Gravid females of Amblyseius fallacies (5) or Phytoseiulus persimilis (9), were picked up at random under a stereomicroscope from leaves taken from plants used to rear the predator colonies. They were transferred individually with a fine camel brush to a small Petri dish (5.5 cm of diameter) containing a leaf piece of the common bean, Phaseolus vulgare. They were treated topically with 0.3 ml of pesticide solution at different dosages using a paintbrush sprayer (Vega 2000, Thayer & Chandler, Lake Bluff, Ill., USA) at 6 psi set at 14.5 cm above the treated area. The pesticide solutions were prepared on the day of application. Treated females were then transferred carefully and individually to each apple leaf disc. To avoid contamination, a new camel brush was used for each concentration to transfer the treated females to leaf discs. Petri dishes were put in a black tray and covered with transparent plastic covers and a strip of brown paper was placed on top to reduce glare and to keep the mites within the leaf disc area. Water was added to the tray to maintain high relative humidity. The trays were incubated in a growth chamber set at 25° C., 75% HR and 16 L Photoperiod. Mortality was recorded 24 h and 48 h after treatment. One and 2 replicates were set up per day respectively for A. fallacis and P. persimilis and only 11 treatments were evaluated for P. persimilis.

Treatments

EC25% is an EC formulation with 25% essential oil as an active ingredient. Seven concentrations of EC25% were prepared as follows. The 1% concentration was prepared by mixing 0.4 ml of the formulation and 9.6 ml of tap water and successive dilutions were made from the stock solution. The following commercially available insecticides were used at their recommended rates: Trounce® (20.2% of fatty acids and 0.2% pyrethrin) at the recommended concentration of 1%; the insect growth regulator Enstar® (s-kinoprene) at the concentration of 0.065%; and Avid® (abamectin 1.9% EC), at the concentrations of 0.0057% and 0.000855%. A water treatment was used as a control for a total of twelve treatments with A. fallacies and 11 with P. persimilis where the Enstar treatment was dropped.

The test product EC25% was sprayed first starting from the lower to the higher concentrations. Then the control treatment was applied followed the reference products Avid, Trounce and Enstar. The spray apparatus was rinsed three times between treatments using successively ethanol 95%, acetone, hexane, distilled water.

Statistical Analysis

Mortality percentages were transformed to logit or probit to determine which analysis gave a better fit as recommended by Robertson and Preisler (1992). The analysis which presents the highest number of small individual Chi square (χ²) is chosen. Probit mortality were regressed on 1+log₁₀ (dose) for EC25%. Concentration mortality regression lines were determined to estimate the lethal concentration to kill 50% of the predator population using the POLO-PC program (LeOra, 1987). Toxicity values of LC₅₀, LC₉₀ and LC₉₉ are given as percent (%) of active ingredient. Data were transformed to arcsine before analysis of variance. Comparison between treatments were analysed using GLM procedure and means were separated by the Fisher test at 5% probability (SAS, 1996).

Results

Amblyseius fallacis

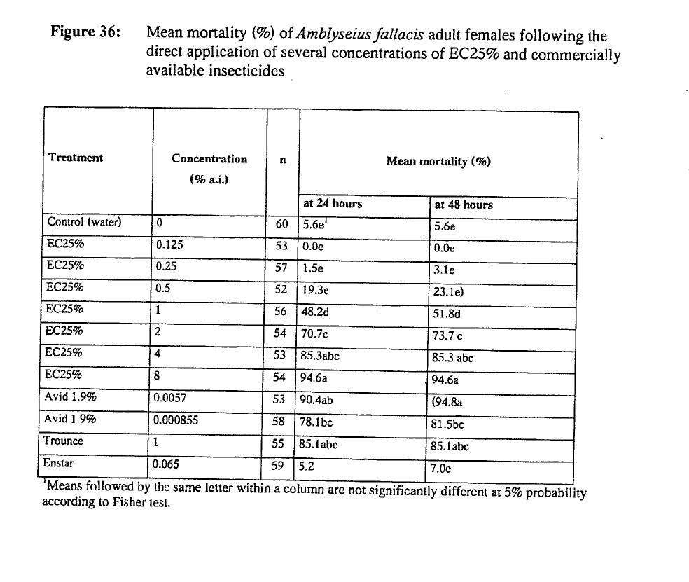

A total of 667 adult females of Amblyseius fallacis was tested and only 12 females (1.79%) walked out of the leaf disc area; number of missing was subtracted from initial total. Mortality in the control was 5.56% at 24 h and remained unchanged at 48 h following treatment (FIG. 36). There was a highly significant difference between treatments at 24 h (F=30.32, df=11, P<0.001) and at 48 h (F=31.64, df=11, P<0.001). There was no mortality after 48 h was with EC25% at the concentration of 0.125% and 3,1% 7% and 23% mortality with EC25% at 0.25%, Enstar and EC25% at 0.5% and these results were not significantly different from the control. Note that at the concentration of 0.5% the EC25% suggested commercial rate, mortality was 23.11% which is less than the 50% limit of the IOBC for harmless pesticides.

Amongst the commercially available products, Trounce caused the highest mortality (85.11%) after 48 H. This was followed by the Avid treatments at concentrations of 0.0057% (94.8% mortality) and 0.000855% (81.5% mortality) and results did not differ significantly, demonstrating that both products are equally toxic to Amblyseius fallacis.

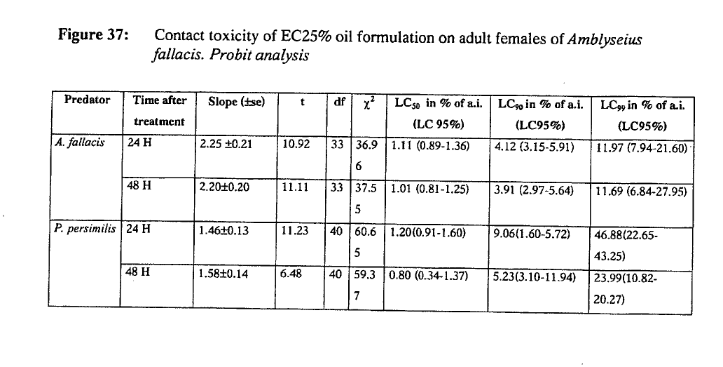

LC₅₀, LC₉₀ and LC₉₉ values at 48 h (FIG. 37) are well above (1.01%, 3.91% and 4.12% respectively) the 0.5% effective dose used to control the spider mite pest, Tetranychus urticae)(Chiasson, unpublished results).

These results indicate that EC25% might have low or no residual toxicity to Ambiyseius fallacis and most adult females that remained alive 24 hours after the EC25% treatments continued to reproduce and were observed laying eggs.

Phytoseiulus persimilis

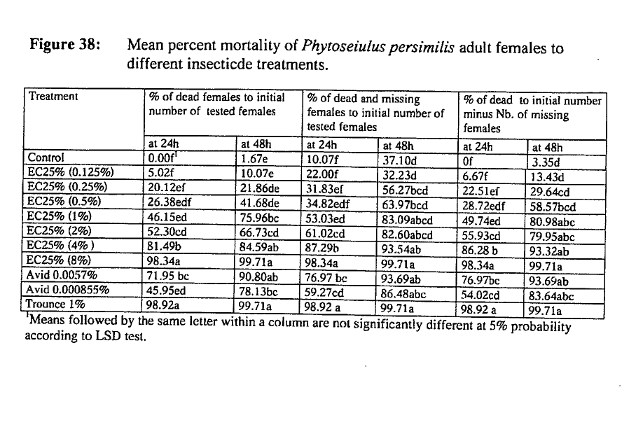

A cohort of 555 adult females was used to evaluate the toxicity of EC25% and the commercially available Trounce and Avid with the mite predator, Phytoseiulus persimilis. In this bioassay, 7.35% and 13.17% of the total number of gravid females escaped from the leaf disc 24 h and 48 h respectively after treatments. They contributed to 13.06% and 18.35% of the total mortality recorded at 24 h and 48 h respectively. The highest number of predator escapees were observed in the control treatment and in the EC25% treatments at concentrations lower than 2%. We will discuss only mortality calculated over total number treated minus missing individuals (3^{rd} column of FIG. 38).

Highest mortality were caused by Trounce (99,71%) followed by Avid at the concentration of 0.0057% (93.69%).The lowest mortality was observed in the treatment with EC25% at the 0.125% concentration (13.43%). Mortality with EC25% at 0.125%, 0.25 and 0.5% were not significantly different from the control treatment.

When missing females were deducted from the initial number of adults tested, the LC₅₀ of P. persimilis was 1.2% and 0.8% at 24 h and 48 h after treatments respectively (FIG. 37).

B. Direct Toxicity of the Essential Oil Extract on Aphid Endoparasitoids Aphidius colemani (Hymenoptera: Brachonidae, Aphidiinae)

In the present study, adult Aphidius colemani wasps were exposed to a direct spray application of EC25% and remained in permanent contact with the biopesticide residues, which is considered worse case conditions, to test the potential side effects this biopesticide may have on beneficial hymenoptera such as Aphidius colemani

Rearing of Aphidius colemani

Aphidius colemani wasps were purchased from.Plant Product Quebec in lots of 250 mixed mummies and adults. The emerged wasps and the remaining mummies were directly transferred to a 5 litre plastic bag filled with air and the wasps were provided with a 10% solution of sucrose and honey (w/w) as food source and water.

Direct Contact Bioassay

Six to 14 adult parasitoids less than 48 h old were transferred into a large solo cup (500 ml ca.) using a mouth aspirator. The solo cup was lined with a filter paper (Rothmans #1) and had two large openings drilled on the side and one on the cover to provide ventilation and these openings were covered with a fine screen to prevent escape of adult wasps and condensation of the pesticide vapour. The filter paper was humidified with a 10% solution of sucrose and honey. The solo cup containing the wasps was weighed and the wasps were dragged down to the bottom of the solo cup by means of successive beats on the cover with a 15 cm long stick. They were treated with 0.3 ml of the insecticide solution using a paintbrush sprayer (Vega 2000, Thayer & Chandler, Lake Bluff, Ill., USA) at 6 psi and set at 14.5 cm above the treated area. The solo cup was then covered and re-weighed to determine weight of pesticide used. The treated wasps were then incubated in a growth chamber set at 18° C.-22° C. and 60-65% HR. Assessment of treatment effects were made at 24 h and 48 h following treatment.

Residual Bioassay

Ten to 20 adult wasps including at least 5 females were picked up and put in a glass Petri dish and covered. The cover had an opening covered with a screen to enable ventilation and to prevent condensation of the pesticide vapour. The Petri dishes were previously treated with a pesticide solution exactly in the same manner as for direct toxicity bioassay but dishes were left to dry for an hour before covering and exposing the wasps to the pesticide residues. On the cover, two small circular holes were drilled and used to provide the wasps with water and a solution of honey and sucrose. Mortality was recorded at 24 h and 48 h.

Treatments

The test product EC25%, a 25% essential oil EC formulation obtained from Codena Inc. Seven concentrations were prepared as follows: EC25% at 8% was prepared by mixing 3.2 ml of EC25% and 6.4 ml of tap water and successive dilutions of 4%, 2%, 1%, 0.5% and 0.125% were made from the stock solution. Commercially available insecticides were used at their respective recommended doses as positive controls: Trounce® (20.2% of fatty acids, Safer Ltd, Scarborough, Ont.) at the recommended concentration of 1%, the insect growth regulator Enstare (s-kinoprene) at the concentration of 0.065%; Avid® (abamectin 1.9% EC) at the concentrations of 0.0057% and 0.000855%, and Thiodan® (endosulfan 50 WP) at the concentration of 5%.

The test product EC25% was used first, starting from the lowest to the highest concentration and followed by the water control and finally by Avid, Trounce, Enstar and Thiodan. The spray apparatus was rinsed three times between treatments using successively ethanol 95%, acetone, hexane, distilled water.

Statistical Analysis

Concentration was analysed as main effect and the weight of pesticide applied was tested as a covariate to correct for difference in quantity of applied pesticide. This covariate was deleted from the model when found not significant. Mortality regression lines were determined to estimate the lethal concentration to kill 10%, 50% and 90% of the parasitoid population using the POLO-PC program (LeOra, 1987). Toxicity values of LC₅₀ are given as percent of active ingredient. Data were transformed to arcsine before analysis of variance but actual means were presented. Comparison between treatments were analysed using GLM procedure and means were separated by Fisher test at 5% probability (SAS, 1996).

Effect of Treatments on Aphidius colemani Emergence from Mummies

Myzus persicae mummies parasitized by Aphidius colemani females on leaves of cabbage (cv. Lennox) were used in this test. Portions of leaves bearing mummies were cut and placed in a Petri dish. The Petri dish was weighted and treated with a pesticide solution and immediately re-weighted to determine the amount of pesticide used. The treated Petri dish was then covered and sealed with parafilm. The cover of the Petri had a screened opening to enable ventilation and to prevent escape of emerging Aphidius adults. The incubation period lasted 7 days and all mummies that did not emergence as adult wasps were considered dead.

Fecundity Assessment

Females that survived the pesticide residual treatments were assessed for fecundity on wheat plants infested with aphids. Myzus persicae aphids reared on cabbage plants (c.v. Lennox) were brushed onto a pot containing 25 to 30 plants of wheat 6 days old. Soon after, the brushed aphids climbed the wheat plants and a density of at least 100 aphids per pot was required. Female wasps that survived the 48 h residual treatments were removed individually from the test arena by means of an aspirator and confined over pots of aphid-infested plants using ventilated transparent plastic cylinders for a period of 24 h. The females were then removed and the plant bearing parasitized aphids were incubated for a period of 10 days at 18° C. to 22° C. At the end of the incubation period, the wheat plant was cut and put in a Petri dish. The number of parasitized aphids were counted.

Results

Direct Contact Bioassay

A total of 1174 adult wasps including 657 or 55.9% female parasitoids were tested.in the bioassay. The mean quantity of pesticide solutions applied was 4.58±1.36 mg/cm which was more than double the amount of 2.0±0.2 mg/cm recommended for the typical bioassay (Mead-Briggs et al., 2000).

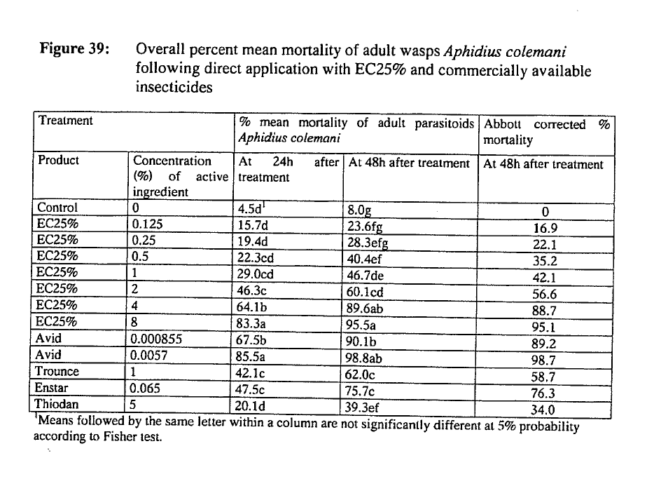

Mortality with EC25% at concentrations up to 1% was not significantly different than for the water control after 24 h. though at 48 h, results with EC25% treatments at the 0.5% and 1% concentrations significantly different from the control (FIG. 39). At the 0.5% concentration of EC25%, recommended for field application, mortality varied from 18.6% to 35.2% at 24 h and 48 H after treatments respectively. Highest mortality was observed with the Avid treatments at concentrations of 0.0057% and 0.000855% and with the EC25% treatment at concentrations of 4% and 8%.

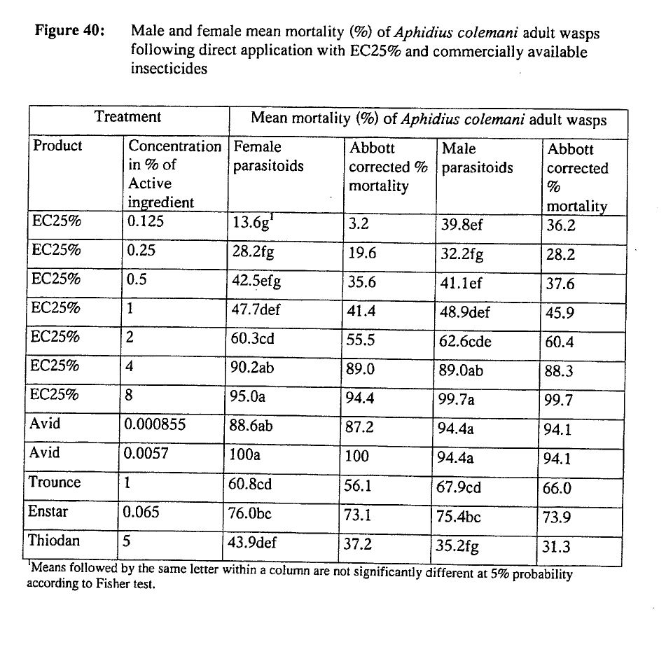

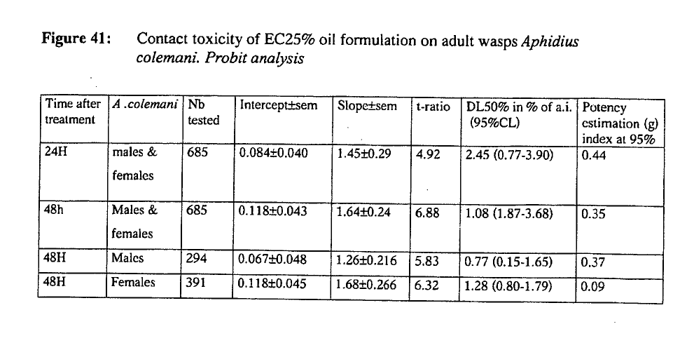

Results in FIG. 40 show that female wasps were relatively less sensitive to treatments than adult males. LC₅₀ values for EC25% on A. colemani females (FIG. 41) was equal to 1.28% which is more than twice the recommended concentration of 0.5% for field application. The LC₅₀ for A. colemani males was lower at 0.77% but still above the 0.5% field recommended concentration of EC25%. However, the 95% confidence limits (CL 95%) of LD50% for both males and females were overlapping and therefore their LD50% were not differently significant (Robertson and Presisler, 1992).

Residual Assay

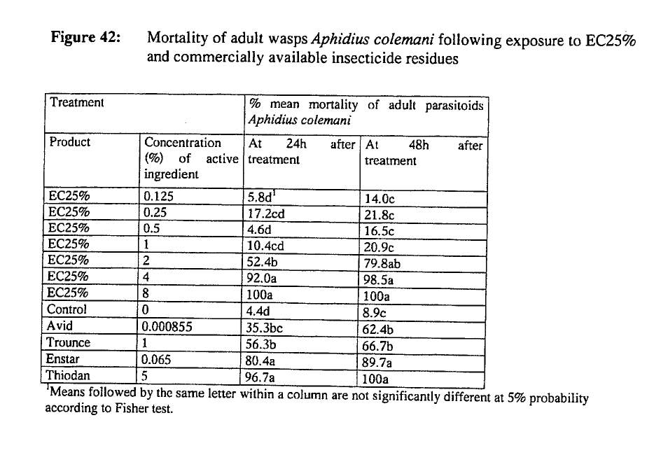

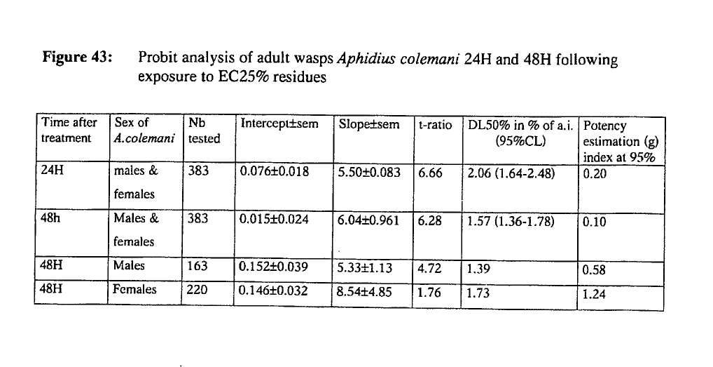

Results shown in FIGS. 42 and 43.

Effect of Treatments on Aphidius colemani Emergence from Treated Mummies

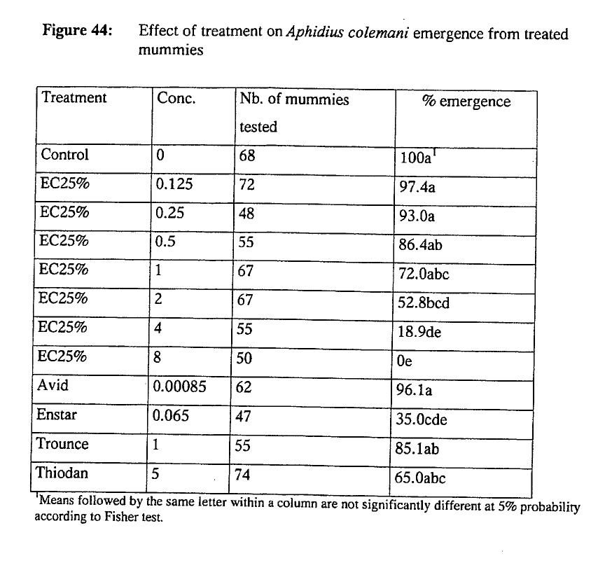

FIG. 44 showed that the effects of treatments on emergence of Aphidius colemani adults from treated mummies were significant (F=6.94, dl=16, P<0.0001). The emergence rate of A. colemani decreased steadily when EC25% concentration increased and there was no emergence at the concentration of 8%. At the recommended concentration for field application, i.e. 0.5%, emergence was 86.4% and this result was not statistically different from that observed in the control. In the reference products tested, the highest emergence was observed in the Avid treatment with 96.1% and the lowest was Enstar at 35% emergence.

Fecundity Assessment

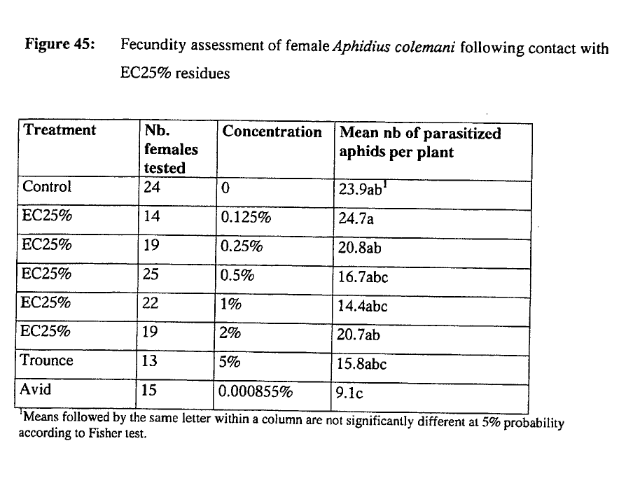

The results of FIG. 45 indicated that females that survived the treatment were able to parasitize Myzus persicae hosts and that their reproductive functions did not seem to be affected. There was no enough surviving female to test for the EC25% concentration of 4% and 8%. The lowest fecundity rate was observed in the treatment of Avid with 9.1 mummies per plants compared to 23.9 mummies per plant recorded in the control treatment. The number of mummies produced from females treated with EC25% treatments at concentrations varying from 0.125 to 2% were not significantly different from the control.

C. Direct Toxicity of the Essential Oil Extract on Predatory Minute Bug Orius insidiosus Say

Various Orius species including Orius insidiosus Say (Heteroptera: Anthocoridae) are effective biological control agents of western flower thrips (WFT) Frankliniella occidentallis Pergrande (Thysanoptera: Thripidae) in sweet pepper, cucumber and other vegetable and ornamental crops (Veire de van et al., 1996).

The present study was initiated to evaluate the side effects of EC25% on the predatory bug Orius insidiosus under laboratory conditions.

Culture of Orius insidiosus

Orius insidiosus stock culture was initiated with individuals obtained from a commercial supplier (Plant Prod Quebec, 3370 Le Corbusier, Laval, Quebec) and maintained in a laboratory growth chamber. Eggs of Ephestia spp were served as a food source and snaps beans of Phaseolus vulgaris as an oviposition substrate. The beans containing eggs were then incubated in folded brown paper until emergence. The folded paper was used to reduce cannibalism. Emerging nymphs were then transferred into one litre jars containing bean pods and fed with Ephestia eggs until the adult stage. The stock culture was renewed regularly.

Direct Contact Bioassay

The bioassays were carried out in small Petri dishes (5.5 cm in dia.) using a leaf disc method. A thin layer of agar 2% (2-3 mm) was poured into each Petri dish and a ring of apple leaf (cv. McIntosh, 3.5 cm in dia.) was cut and placed upside down on the surface of the agar.

At least 10 Orius insidiosus 2^{nd} nymph instar or adults were transferred carefully using an aspirator on the surface of the apple leaf disc. The Petri dish containing the nymphs or the adults bugs were dragged down to the bottom of the Petri dish by means of successive beats on the cover with a 15 cm long stick. The Petri dishes were weighted and immediately, they were treated immediately with 0.3 ml of pesticide solution at different concentrations using a paintbrush sprayer (Vega 2000, Thayer & chandler, Lake Bluff, Ill., USA) at 6 psi and set at 14.5 cm above the treated area. The Petri dishes were then re-weighted to determine the quantity of pesticide applied. The pesticide solutions were prepared on the day of treatment. The treated nymphs or adults were then transferred carefully to the surface of the apple leaf disc containing eggs of Ephestia spp as a source of food. To avoid contamination, a new camel brush is used for each concentration to transfer the treated nymphs or adults to the leaf discs. The Petri dishes were put in a tray and incubated in a growth chamber set at 25° C., 65% HR and 16 L Photoperiod. A fan was placed in front of the tray to provide continuous air flow. Mortality of nymphs was recorded at 1, 2, 5, 7 and 9 days after treatment when more than 80% of the nymphs became adults. Mortality of adult predators was recorded at 24 H and 48 H following treatment. Ten replicates were prepared per treatment and 12 treatments were evaluated on second instar nymphs and adults.

Treatments

The test product is EC25%, a 25% EC essential oil formulation obtained from Codena Inc. Seven concentrations were prepared as follow: EC25% at 8% was prepared by mixing 3.2 ml of EC25% and 6.4 ml of tap water and successive dilutions of 4%, 2%, 1%, 0.5% and 0.125% were made from the stock solution. EC25% was compared to the recommended doses of the following commercially available insecticides: Trounce® (20.2% potassium salts of fatty acids and 0.2% pyrethrins) at the recommended concentration of 1%; the insect growth regulator Enstar® (S-kinoprene), at the recommended concentration of 0.065% and Avide® (abamectin 1.9% EC) at the concentration of 0.000855%, Thiodane® (endosulfan 50 WP) at the concentration of 5% and Cygon® (dimethoate) at the concentration of 4%. Water was used as a negative control.

The test product EC25% was sprayed first, starting from the lowest to the highest concentration followed by the water control treatment and finally by the reference products Avid, Cygon, Enstar, Thiodan and Trounce. The sprayer was rinsed three times between treatments using successively ethanol 95%, acetone, hexane and distilled water.

Fecundity Assessment

The potential sublethal effects of EC25% on Orius insidiosus female fecundity was monitored. Fecundity assessment was carried out on females that survived 48 h after the direct contact pesticide treatments. Surviving females were separated from males and put individually in a Petri dish filled with a 2 mm layer of agar used as a support and an apple ring (5.5 cm) placed upside down on the agar surface along with a 3 cm long pod of faba bean (Phaseolus vulgare). The apple leaf disc and the bean pod were used as oviposition substrates. The Petri dish was covered with the correspondent cover and sealed with parafilm. The Petri cover had an opening covered with fine muslin tissue for ventilation and air exchange. Females were left undisturbed for 48 H for oviposion and then were fed with sufficient numbers of Ephestia spp eggs. After the 48 h period, females were then transferred to another Petri dish for a second 48 H oviposition test. During both periods, the eggs laid were counted and left to hatch for 5 days. The eggs that do not hatch after 5 days were considered dead and not viable.

Statistical Analysis

LC₅₀ values of EC25% were determined using probit analysis with POLO software (LeOra, 1987). Concentrations were analysed as main effects and the weight of pesticide applied was tested as a covariance to correct for difference in quantity of the applied pesticide. This covariance was deleted from the model when found not significant. Mortalities were analysed using General Linear model (GLM) procedure within SAS (SAS, 1996) and the number of individuals initially introduced were tested as a covariant. Means were adjusted for covariance when appropriate and separated using the Fisher test for means comparison. However, actual means were presented in the results section.

Results

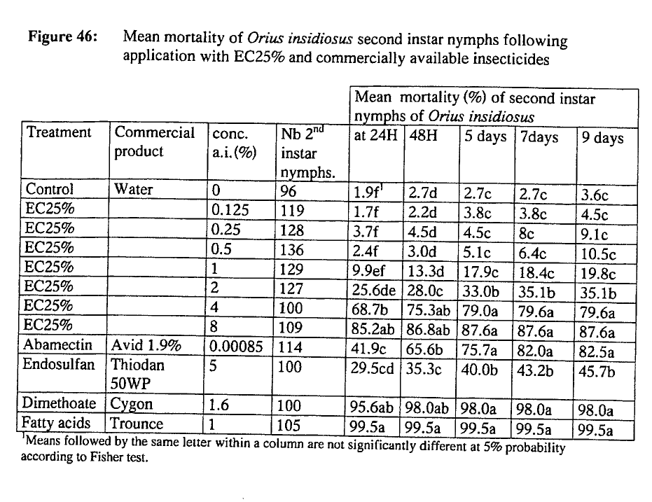

Results show (FIG. 46) that nine days following treatment application, with Orius nymphs, the most toxic treatments were in decreasing order, Trounce (99,5% mortality), Cygon (98% mortality), EC25% at 8% concentration (87.6% mortality), Avid (82.5% mortality) and EC25% at 4% concentration (79.6% mortality). All results were significantly different from that of the control treatment (3.6% mortality). Less than 50% mortality was obtained with the other treatments though only Thiodan (45.7%) and EC25% (35.1%) results were significantly different from the control. Results with EC25% at the recommended concentration for field application of 0.5% were not significantly different from results obtained with the control.

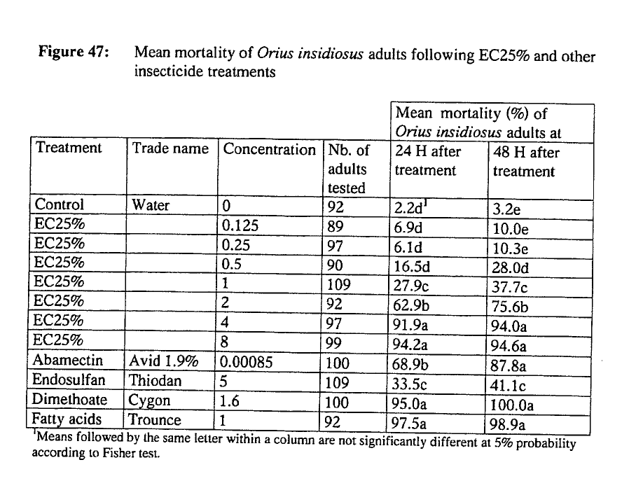

Results show (FIG. 47) that the effects of the 12 treatments expressed as percent mortality of adults was significantly different at 24 H after treatment (F=55.9, df=11, p<0.0001) and at 48 H after treatment (F=63.2, df=11, p<0.0001). The least toxic treatments of EC25% at concentration of 0.125% and 0.25% were not statistically different from the control treatment. The treatment of EC25% at the recommended field concentration of 0.5% was the least toxic of the remaining treatments causing a mortality of 28%. The most toxic group included Cygon (100% mortality), Trounce (98.9% mortality), EC25% at concentrations of 4 and 8% (94% and 94% respectively) and Avid (87.8%).

Fecundity Assessment

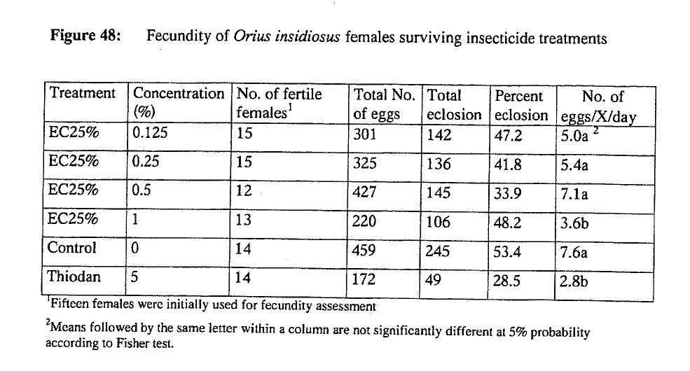

The results from the fedundity assessment assay (FIG. 48) showed that almost all females tested had laid eggs. There were few surviving females to test for fecundity following treatments with Avid, Cygon, Trounce and EC25% at concentrations of 2, 4 and 8%. The mean number of eggs laid per female per day in the control treatment was 7.6 which was almost 4 times the minimum number of 2 eggs per female per day set by the IOBC standards for fecundity for Orius leavigatus, a closely related species of O. insidiosus. The lowest rate was 2.8 eggs per female per day obtained in the treatment with Thiodan followed by EC25% at 0.5% concentration with 3.6 eggs per female per day and both were significantly different from the rate obtained with the water control (7.6 eggs per female per day). The date rate of eggs laid in the EC25% treatments at concentrations of 0.25% (5 eggs) and 0.5% (5.4 eggs) were not significantly different from the number of eggs laid in the control. The eclosion rate varied from 28.5% in the Thiodan treatment to 53% in the control. There was 33.9% egg eclosion in the EC25% at 0.5% concentration treatment.

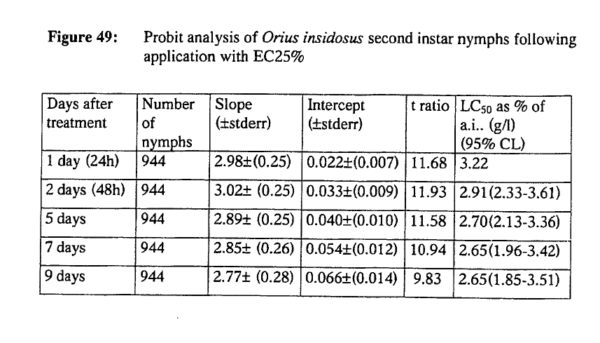

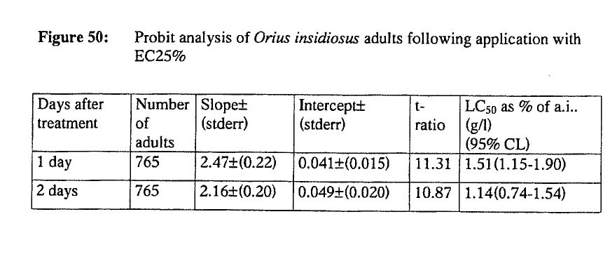

LC₅₀ values for Orius nymphs were 2.65%, 9 days following treatment with EC25% (FIG. 49) and for adults were 1.14%, 2 days following treatment with EC25% (FIG. 50).

References:

Ascher, K. R. S. and Cwilich, R. (1960) Laboratory Evaluation of Acaricides Against Tetranychus Telarius L. on Sugar Beet and on Beans. Israel J Agric. Res. 10:159-163.
ASTM. 1987. Standard test methods for moisture, ash, and organic matter of peat and other organic soils. American Standards for Testing and Materials (ASTM) D 2974-87.
ASTM. 1989. Standard test method for pH of soils. American Standards for Testing and Materials (ASTM) D 4972-89.
Bélanger et al., (1991) Extraction et Détermination de Composés Volatils de L'ail (Allium sativum), Riv. Ital. EPPOS 2:455-461.
Busvine, J. R. (1980) Recommended Methods for Measurement of Pest Resistance to Pesticides. FAO Plant Production and Protection Paper. pp. 49-53.
Ceske, L. J. and P. B. Kaufman, (1999) Regulation of Metabolite Synthesis in Plants. In: Natural Products from Plants. Kaufman et al., eds. CRC Press. Boca Raton. pp. 91-121.
Chialva et al., (1983) Chemotaxonomy of Wormwood (Artemisia Absinthum L.). Z. Lebensm Unters Forsch 176:363-366.
Dittrich, V. (1962) A Comparative Study of Toxicological Test Methods on a Population of the Two-spotted Spider Mite (Tetranychus telarius). J. Econ. Entomol. 55:644-648.
Duerbeck, K., et al., (1997) The Distillation of Essential Oils. Manufacturing and Plant Constuction Handbook. Protrade: Dept. of Foodstuffs and Agricultural Products. Eschborn, Germany. pp. 21-25.
Ebeling, W. and R. J. Pence. (1953) Pesticide Formulation: Influence of Formulation on Effectiveness. J. Agr. Food Chem. 1:386-397.
Feng, R. and M. B. Isman. (1995) Selection for resistance to azadirachtin in the green peach aphid, Myzus persicae, Experientia 51:831-833.
Flint, M. L. (1990) Pests of the Garden and Small Farm. A Grower's Guide to Using Less Pesticide. Statewide IPM Project. U. of California. Publication 3332. pp. 116-123.
Foott, W. H. and H. R. Boyce. (1966) A Modification of the Leaf-Disc Technique for Acaricide Tests. Proc. Entomol. Soc. Ont. 96:117-119.
Georghiou, G. P. (1990) Overview of Insecticide Resistance. In: Managing Resistance to Agrochemicals. M. E. Green, H. M. Le Baron and W. K. Moberg, eds., ACS Symposium Series. Chapter 2. Vol. 421:18-41.
Gundersin et al., (1985). Effects of the Mint Monoterpene Pulegone on Spodoptera Eridania (Lepidoptera: Noctuidae) Environ. Entomol. 14:859-863.
Isman, M. (1999) Pesticides Based on Plant Essential Oils. Pesticide Outlook. pp. 68-72.
Jackson, S. A. L. and R. K. M. Hay. (1994) Characteristics of varieties of thyme (Thymus vulgaris L.) for use in the UK: Oil content, composition and related characters. J. Hort. Sci. 69:275-281.
LeOra Software. POLO-PC, (1994) Probit or Logit Analysis. LeOra Software, Berkeley Calif. pp. 1-28
Lippold, P. (1963) Acaricidal Testing Techniques in the Two-spotted Spider Mite. Adv. Acarol. 1:174-180.
Mead-Briggs, et al., (2000) A laboratory test for evaluating the effects of plant protection products on the parasitic wasp, Aphidius rhopalosiphi (Destephani-Perez)(Hymenoptera: Braconidae). Pages 13-25. In. Guidelines to evaluate side effects of plants protection products to non target arthropods, IOBC, BART and EPPO Joint Initiative. Candolfi, M. P., Blumel, S., Forster, R. Bakker, F. M., Grimm, C., Hassan, S. A., Heimbach, U., Mead-Briggs, M. A., Reber, B, Schmuck, R., Vogt, H. (Edts.), Reinham, Germany.
Meister, R. T. ed. (1999) Farm Chemicals Handbook. Vol. 85. Meister Publishing Company, Willoughby, Ohio.
Perez- Souto, N. et al., (1992) Use of high-performance liquid chromatographic peak deconvolution and peak labeling to identify antiparasitic components in plant extracts. J. of Chrom. 593:209-215.
Rembald, H. (1989) Azadirachtins. Their Structure and Mode of Action. In: Insecticides of Plant Origin. ACS Symposium Series No. 387. pp. 150-163.
Roush, R. T. and J. A. McKenzie. (1987) Ecological Genetics of Insecticide and Acaricide Resistance. Ann. Rev. Entomol. 32:361-380
The McGraw-Hill Dictionary of Chemical Terms (ed. Parker, S., 1985), McGraw-Hill, Sam Francisco.
U.S. Pat. No. 4,933,371
U.S. Pat. No. 5,839,224

1. An essential oil extract derived from a Chenopodium species, comprising: from at least 30% to at least 65% α-terpinene, from at least 8% to at least 26% ρ-cymene, from at least 5% to at least 24% limonene, from at least 0.04% to at least 1% carvacrol, from at least 0.1% to at least 2% carveol, from at least 0.1% to at least 2.0% nerol, from at least 0.04% to at least 2% thymol, and from at least 0.04% to at least 1% carvone, and wherein said extract is active against one or more pests chosen from insects, acari, and fungi.

2. (Canceled)

3. (Canceled)

4. (Canceled)

5. The essential oil extract according to claim 1, wherein said species is Chenopodium ambrosioides.

6. A pesticidal composition, comprising an effective amount of the essential oil extract of claim 1 and a suitable emulsifier, spreader or sticking agent, and carrier.

7. The pesticidal composition according to claim 6, comprising between 0.125% to 10% (by volume) essential oil extract.

8. The pesticidal composition according to claim 7, comprising between 0.25% to 5% (by volume) of said essential oil extract.

9. The pesticidal composition according to claim 6, wherein said composition comprises between 5% to 50% (by volume) of said essential oil extract.

10. The pesticidal composition according to claim 9, comprising between 10% to 25% (by volume) of said essential oil extract.

11. The pesticidal composition according to claim 10, comprising between 1% to 15% (by volume) of a suitable emulsifier, and between 50% to 70% (by volume) of a suitable carrier or solvent.

12. The pesticidal composition according to claim 11, comprising between 2% to 20% (by volume) of a suitable spreader or sticking agent.

13. A pesticidal composition, comprising between 30% to 50% (by volume) of the essential oil extract of claim 1, between 0.5% to 25% (by volume) of a suitable emulsifier, and between 10% to 50% (by volume) water.

14. A method for controlling one or more pests chosen from phytophagous acari, phytophagous insects, and phytophagous fungi, which comprises applying to a locus where control is desired a pesticidally-effective amount of the pesticidal composition of claim 6.

15. (Canceled)

16. (Canceled)

17. The method according to claim 14, wherein said locus is soil.

18. The method according to claim 14, wherein said locus is a plant.

19. The essential oil extract according to claim 1, further comprising ascaridole at a proportion of 9.86% or less.

20. The essential oil extract according to claim 1, further comprising ascaridole at a proportion of 1% or less.

21. The essential oil extract according to claim 20, wherein said extract has trace or undetectable amounts of ascaridole.

22. A formulation for indoor or outdoor fumigation, comprising: between 1% to 15% (by volume) of the essential oil extract of claim 1.

23. A formulation for topical application, comprising: between 1% to 10% (by volume) of the essential oil extract of claim 1.

24. The formulation of claim 23, wherein the formulation further comprises between 2% to 5% (by volume) of an emollient, between 0.1% to 1% (by volume) of a stabilizer and between 0.1% to 0.5% (by volume) of a preservative.

25. The formulation of claim 23, wherein the formulation further comprises between 1% to 10% (by volume) of an emulsifier, between 2% to 5% (by volume) of a coat or skin conditioner, between 0. 1% to 1% (by volume) of a stabilizer, between 0. 1% to 1% (by volume) of a preservative and between 0. 1% to 0.5% (by volume) of an antioxidant.

26. A microemulsion formulation, comprising: between 30% to 50% (by volume) of the essential oil extract of claim 1, between 0.5% to 25% (by volume) of a suitable emulsifier, and between 10% to 50% (by volume) water.

27. The formulation of claim 26, wherein said formulation is for soil delivery.

28. A method for controlling phytophagous pests, which comprises applying to a locus where control is desired a pesticidally-effective amount of the formulation according to any one of claims 22, 23, or 26.
